(19) United States
(12) Patent Application Publication
(10) Pub. No.: US 2025/0216407 A1
(43) Pub. Date: Jul. 3, 2025

(54) LIVER DISEASE TREATMENT AND ASSESSMENT USING OXYLIPINS

(71) Applicant: Inipharm, Inc., Bellevue, WA (US)

(72) Inventors: Heather Kay Webb HSU, Seattle, WA (US); Cindy MCREYNOLDS, Davis, CA (US)

(21) Appl. No.: 18/850,234

(22) PCT Filed: Mar. 28, 2023
(86) PCT No.: PCT/US2023/016616

§ 371 Date: Sep. 24, 2024

Related U.S. Application Data
(60) Provisional application No. 63/370,347, filed on Aug. 3, 2022.
(60) Provisional application No. 63/324,882, filed on Mar. 29, 2022.

(51) Int. Cl. G01N 33/92

(52) U.S. Cl. CPC G01N 33/92; G01N 2800/085; G01N 2800/52

(57) ABSTRACT

Provided are biomarker panels and methods of using oxylipins as biomarkers. Some embodiments include methods for treating a patient based on oxylipin measurements obtained in the subject. The oxylipin measurements may be used to inform use of an HSD17B13 inhibitor in the subject.

### CROSS-REFERENCE

This application claims the benefit of U.S. Provisional Application No. 63/324,882 filed Mar. 29, 2022, and U.S. Provisional Application No. 63/370,347 filed Aug. 3, 2022, which are incorporated herein by reference in their entireties.

### BACKGROUND

Liver diseases pose a serious problem to many individuals. There is a need for improved methods for evaluating and treating liver diseases.

### SUMMARY

Disclosed herein, in some embodiments, are treatment methods, comprising: obtaining or receiving an oxylipin measurement in a biological sample of a subject; and based on the oxylipin measurement, administering or discontinuing administration of a 17β-hydroxysteroid dehydrogenase type 13 (HSD17B13) inhibitor to the subject. Some embodiments further include identifying the subject as a candidate for HSD17B13 inhibitor treatment based on the oxylipin measurement. In some embodiments, the subject has already received an earlier HSD17B13 inhibitor administration. Some embodiments further include identifying an effective dose of the HSD17B13 inhibitor based on the oxylipin measurement. Some embodiments further include identifying a dose of the HSD17B13 inhibitor based on the oxylipin measurement that reproduces a HSD17B13 phenotype in the subject. In some embodiments, administering the HSD17B13 inhibitor to the subject comprises adjusting a dose of the HSD17B13 inhibitor. Some embodiments further include evaluating a HSD17B13 inhibitor treatment of the subject based on the oxylipin measurement. Some embodiments further include evaluating a disease or likelihood of disease development in the subject state based on the oxylipin measurement. In some embodiments, the oxylipin measurement is elevated or is decreased in the subject, relative to a control or baseline oxylipin measurement. In some embodiments, the oxylipin comprises an oxylipin in Table 1. In some embodiments, the oxylipin comprises 9(10)-EpO, 10-Nitrooleate, 9-Nitrooleate, 9,10-e-DiHO, 9(10)-EpOME, 12(13)-EpOME, 10-Nitrolinoleate, 9-KODE, 13-KODE, 12(13)Ep-9-KODE, 9-HODE, 13-HpODE, 9-HpODE, 9,12,13-TriHOME, 9,10-DiHOME, 12,13-DiHOME, 9(10)-EpODE, 15(16)-EpODE, 12(13)-EpODE, 9-HOTE, 13-HOTE, 9,10-DiHODE, 15,16-DiHODE, 12,13-DiHODE, PGE1, 8,15-DiHETE, 5,15-DiHETE, LTB4, 8(9)-EpETrE, 14(15)-EpETrE, 11(12)-EpETrE, PGD2, 6-keto-PGF1a, 15-keto-PGE2, 15-deoxy PGJ2, PGE2, PGF2a, 15-KETE, 5-KETE, 8-HETE, 9-HETE, 11-HETE, 5-HETE, 20-HETE, LxA4, LxB4, TXB2, 14,15-DiHETrE, 11,12-DiHETrE, 8,9-DiHETrE, 5,6-DiHETrE, RvE2, LTB5, 17(18)-EpETE, 14(15)-EpETE, 11(12)-EpETE, PGE3, PGF3a, 9-HEPE, 5-HEPE, 18-HEPE, 15-HEPE, 12-HEPE, 17,18-DiHETE, 14,15-DiHETE, Mar-1, PDX, 19(20)-EpDoPE, 16(17)-EpDoPE, 4-HDoHE, 17-HDoHE, 14-HDoHE, RvD2, 19,20-DiHDoPE, PEA, SEA, OEA, LEA, aLEA, AEA, DEA, PGE2 EA, DHEA, PGF2a EA, PGD2 EA, 15-HETE EA, or 11(12)-EpETre EA. In some embodiments, the oxylipin comprises 9(10)-EpO, 10-Nitrooleate, 9-Nitrooleate, 9,10-e-DiHO, 9(10)-EpOME, 12(13)-EpOME, 10-Nitrolinoleate, 9-KODE, 13-KODE, 12(13)Ep-9-KODE, 9-HODE, 13-HpODE, 9-HpODE, 9,12,13-TriHOME, 9,10-DiHOME, 12,13-DiHOME, 9(10)-EpODE, 15(16)-EpODE, 12(13)-EpODE, 9-HOTE, 13-HOTE, 9,10-DiHODE, 15,16-DiHODE, 12,13-DiHODE, PGE1, 8,15-DiHETE, 5,15-DiHETE, LTB4, 8(9)-EpETrE, 14(15)-EpETrE, 11(12)-EpETrE, PGD2, 6-keto-PGF1a, 15-keto-PGE2, 15-deoxy PGJ2, PGE2, PGF2a, 15-KETE, 5-KETE, 8-HETE, 9-HETE, 11-HETE, 5-HETE, 20-HETE, LxA4, LxB4, TXB2, 14,15-DiHETrE, 11,12-DiHETrE, 8,9-DiHETrE, 5,6-DiHETrE, RvE2, LTB5, 17(18)-EpETE, 14(15)-EpETE, 11(12)-EpETE, PGE3, PGF3a, 9-HEPE, 5-HEPE, 18-HEPE, 15-HEPE, 12-HEPE, 17,18-DiHETE, 14,15-DiHETE, 19(20)-EpDoPE, 16(17)-EpDoPE, 4-HDoHE, 17-HDoHE, 14-HDoHE, RvD2, 19,20-DiHDoPE, PEA, SEA, OEA, LEA, aLEA, AEA, DEA, PGE2 EA, DHEA, PGF2a EA, PGD2 EA, 15-HETE EA, 11(12)-EpETre EA, 20-COOH-LTB4, 20-OH-LTB4, PGD3, 9,10,13-TriHOME, PGD1, 11,12-,15-TriHETrE, PGJ2, PGB2, 6-trans-LTB4, 11,12-DiHETE, 8,9-DiHETE, 19,20-DiHDPE, 16,17-DiHDPE, 13,14-iHDPE, 9-HOTrE, 10,11-DiHDPE, EKODE, 13-HOTrE, 5,6-DiHETE, 15-deoxy-PGJ2, 7,8-DiHDPE, 8-HEPE, 4,5-DiHDPE, 13-oxo-ODE, 15-oxo-ETE, 9-oxo-ODE, 8(9)-EpETE, 15(S)-HETrE, 12-oxo-ETE, 19(20)-EpDPE, 16(17)-EpDPE, 13(14)-EpDPE, 5-oxo-ETE, 10(11)-EpDPE, 7(8)-EpDPE, 8(9)-EpETrE, alt 5(6)-EpETrE, LTC4, LTD4, or LTE4. In some embodiments, the oxylipin comprises an epoxide oxylipin. In some embodiments, the oxylipin comprises a hydroxyl oxylipin. In some embodiments, the oxylipin comprises a keto oxylipin. In some embodiments, the oxylipin is arachidonic acid-derived. In some embodiments, the oxylipin is eicosapentaenoic acid (EPA)-derived. In some embodiments, oxylipin is docosahexaenoic acid (DHA)-derived. In some embodiments, the oxylipin is linoleic acid-derived. In some embodiments, the oxylipin is alpha-linoleic acid-derived. In some embodiments, the oxylipin is endogenous to the subject. In some embodiments the oxylipin is in a free fatty acid form. In some embodiments the oxylipin is in an esterified form. In some embodiments, the oxylipin comprises an oxylipin other than 13-HODE, 15-HETE, 12-HETE, RvE1, RvD1, LTB3, PDX, or Mar-1, or a combination thereof. In some embodiments, the oxylipin comprises an oxylipin other than 13-HODE, 15-HETE, 12-HETE, RvE1, RvD1, LTB3, PDX, and Mar-1. Some embodiments further include administering linoleic acid to the subject prior to obtaining the oxylipin measurement, or wherein the subject has been administered linoleic acid prior to receiving the oxylipin measurement. In some embodiments, the administered linoleic acid comprises a label. Some embodiments further include administering alpha-linoleic acid to the subject prior to obtaining the oxylipin measurement, or wherein the subject has been administered alpha-linoleic acid prior to receiving the oxylipin measurement. In some embodiments, the administered alpha-linoleic acid comprises a label. In some embodiments, the label comprises an isotopic label. In some embodiments, the isotopic label comprises ¹³C or ²H. In some embodiments, the oxylipin comprises the label. In some embodiments, the oxylipin measurement comprises 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, or 132 oxylipin measurements. In some embodiments, the oxylipin measurement comprises measurements of at least: 2, 3, 4, 5, 6, 7, 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, or 90 different oxylipins. In some embodiments, the disease comprises a liver disease. In some embodiments, the sample comprises a tissue. In some embodiments, the tissue comprises liver sample. In some embodiments, the sample comprises a biofluid sample. In some embodiments, the biofluid comprises blood, serum, or plasma. In some embodiments, the sample comprises a sample of lipoproteins. In some embodiments, the lipoproteins comprise low-density lipoproteins (LDLs), very low-density lipoproteins (VLDLs), or high-density lipoproteins (HDLs). In some embodiments, the lipoproteins are enriched in the sample. In some embodiments, the subject is a mammal. In some embodiments, the subject is a human.

### BRIEF DESCRIPTION OF THE DRAWINGS

FIG. 1A-1C show data that demonstrates the effect of dosing Zucker obese rats with a HSD17B13 inhibitor on the presences of 4 oxylipins, 5-HEPE, 12-HETE, 9-HETE, and 13-HODE. The data for FIG. 1A was collected at day three and tested for free oxylipins, FIG. 1B was collected on day 21 and tested for free oxylipins, and FIG. 1C was collected was collected on day 21 and tested for both free and esterified oxylipins.

FIG. 2 displays data that demonstrates the effect on free versus esterified HSD substrates in Zucker obese rats treated for 21 days with a HSD17B13 inhibitor.

FIG. 3A-3C display results of a hierarchical cluster analysis which identified 6 clusters of bioactive lipids that segregated together. Cluster 4 which contained known HSD17B13 substrates was significantly elevated in response to HSD17B13 inhibitor dosing and was composed dominantly with esterified oxylipins.

### DETAILED DESCRIPTION

The HSD17B13 gene encodes the oxidoreductase, 17β-Hydroxysteroid dehydrogenase type 13 (HSD17B13). Inactive polymorphisms of the HSD17B13 gene may be protective against chronic liver diseases including alcoholic liver disease (ALD) and non-alcoholic steatohepatitis (NASH). It may be useful to identify biomarkers associated with HSD17B13 activity or associated with target engagement by inhibitors of HSD17B13. Oxylipins may be useful as biomarkers for such purposes. In some cases, genotype-specific changes in oxylipin metabolism may be used as target engagement a biomarker in subjects receiving HSD17B13 inhibitor treatment. A panel of oxylipins may be used as a signature or indicator of HSD17B13 activity.

Provided herein are methods of treatment. The method may include obtaining or receiving an oxylipin measurement in a biological sample of a subject; and based on the oxylipin measurement, administering or discontinuing administration of a 17β-hydroxysteroid dehydrogenase type 13 (HSD17B13) inhibitor to the subject. Some embodiments include identifying the subject as a candidate for HSD17B13 inhibitor treatment based on the oxylipin measurement, identifying an effective dose of the HSD17B13 inhibitor based on the oxylipin measurement, identifying a dose of the HSD17B13 inhibitor based on the oxylipin measurement that reproduces a HSD17B13 phenotype (e.g. an inactive or semi-active HSD17B13 phenotype) in the subject, evaluating a HSD17B13 inhibitor treatment of the subject based on the oxylipin measurement, or evaluating a disease or likelihood of disease development in the subject state based on the oxylipin measurement.

### Oxylipin Biomarkers

Disclosed herein, in some embodiments are biomarkers comprising oxylipins. The biomarkers may be used in a method described herein. For example, some methods may include obtaining or receiving oxylipin measurements, and using them for a variety of purposes.

Some examples of oxylipins are included in Table 1. The oxylipins in Table 1 may be used as biomarkers, for example as indicators of HSD17B13 function. Table 2 includes information on some aspects of the oxylipins in Table 1.

TABLE 1
Examples of Oxylipins

| Oxylipin Name | Abbreviation(s) |
|---|---|
| 9(10) epoxysteric acid | 9(10)-EpO |
| 10-Nitrooleate | 10-Nitrooleate |
| 9-Nitrooleate | 9-Nitrooleate |
| 9,10 dihydroxysteric acid | 9,10-e-DiHO |
| 9,10-epoxy-octadecenoic acid | 9(10)-EpOME |
| 12(13)epoxy-octadecenoic acid | 12(13)-EpOME |
| 10-Nitrolinoleate | 10-Nitrolinoleate |
| 9-oxo-octadecadienoic acid | 9-KODE; 9-osoODE |
| 13-oxo-octadecadienoic acid | 13-KODE; 13-oxoODE |
| 9-oxo-12,13-Epoxy-10-octadecenoate | 12(13)Ep-9-KODE |
| 13-hydroxy-octadecadienoic acid | 13-HODE |
| 9-hydroxy-octadecadienoic acid | 9-HODE |
| 13-hydroperoxy-octadecadienoic acid | 13-HpODE |
| 9-hydroperoxy-octadecadienoic acid | 9-HpODE |
| 9,12,13-trihydroxy-octadecenoic acid | 9,12,13-TriHOME |
| 9,10-dihydroxy-octadecenoic acid | 9,10-DiHOME |
| 12,13-dihydroxy-octadecenoic acid | 12,13-DiHOME |
| 8-{3-[octa-2,5-dien-1-yl]oxiran-2-yl}octanoic acid | 9(10)-EpODE |
| 14-(3-ethyloxiran-2-yl)tetradeca-9,12-dienoic acid | 15(16)-EpODE |
| 11-{3-[pent-2-en-1-yl]oxiran-2-yl}undec-9-enoic acid | 12(13)-EpODE |
| 9-hydroxyoctadeca-9,11,15-trienoic acid | 9-HOTE |
| 13-hydroxyoctadeca-9,11,15-trienoic acid | 13-HOTE |
| 9,10-dihydroxyoctadeca-12,15-dienoic acid | 9,10-DiHODE |
| 15,16-dihydroxyoctadeca-9,12-dienoic acid | 15,16-DiHODE |
| 12,13-dihydroxyoctadeca-9,15-dienoic acid | 12,13-DiHODE |
| Prostaglandin E1 | PGE1 |
| 8,15-dihydroxy-eicosatetraenoic acid | 8,15-DiHETE |
| 5,15-dihydroxy-eicosatetraenoic acid | 5,15-DiHETE |
| Leukotriene B4 | LTB4 |
| 8,9-epoxy-eicosatrienoic acid | 8(9)-EpETrE; 8(9)EET |
| 14(15)-epoxy-eicosatrienoic acid | 14(15)-EpETrE; |
| | 14(15)EET |
| 11,(12)-epoxy-eicosatrienoic acid | 11(12)-EpETrE; |
| | 11(12)EET |
| Prostaglandin D1 | PGD2 |
| 6-keto Prostaglandin F1α | 6-keto-PGF1a |
| 15-keto Prostaglandin E2 | 15-keto-PGE2 |
| 15-keto Prostaglandin J2 | 15-deoxy PGJ2 |
| Prostaglandin E2 | PGE2 |
| Prostaglandin E2 | PGF2a |
| 15-oxo-eicosatetraenoic acid | 15-KETE; 15-oxoETE |
| 5-oxo-eicosatetraenoic acid | 5-KETE; 5-oxoETE |
| 8-hydroxy-eicosatetraenoic acid | 8-HETE; 8-oxoETE |
| 9-hydroxy-eicosatetraenoic acid | 9-HETE; 9-oxoETE |
| 15-hydroxy-eicosatetraenoic acid | 15-HETE; 15-oxoETE |
| 12-hydroxy-eicosatetraenoic acid | 12-HETE; 12-oxoETE |
| 11-hydroxy-eicosatetraenoic acid | 11-HETE; 11-oxoETE |
| 5-hydroxy-eicosatetraenoic acid | 5-HETE; 5-oxoETE |
| 20-hydroxy-eicosatetraenoic acid | 20-HETE; 20-oxoETE |
| Lipoxin A4 | LxA4 |
| Lipoxin B4 | LxB4 |
| Thromboxane B2 | TXB2 |
| 14,15-dihydroxy-eicosatrienoic acid | 14,15-DiHETrE |
| 11,12-dihydroxy-eicosatrienoic acid | 11,12-DiHETrE |
| 8,9-dihydroxy-eicosatrienoic acid | 8,9-DiHETrE |
| 5,6-dihydroxy-eicosatrienoic acid | 5,6-DiHETrE |
| Resolvin E2 | RvE2 |
| Leukotriene B5 | LTB5 |
| 17,18-epoxy-eicosatetraenoic acid | 17(18)-EpETE |
| 14,15-epoxy-eicosatetraenoic acid | 14(15)-EpETE |
| 11,12-epoxy-eicosatetraenoic acid | 11(12)-EpETE |
| Prosteglandin E3 | PGE3 |
| Prosteglandin F3a | PGF3a |
| 9-hydroxy-eicosapentaenoic acid | 9-HEPE |
| 5-hydroxy-eicosapentaenoic acid | 5-HEPE |
| 18-hydroxy-eicosapentaenoic acid | 18-HEPE |
| 15-hydroxy-eicosapentaenoic acid | 15-HEPE |
| 12-hydroxy-eicosapentaenoic acid | 12-HEPE |
| Resolvin E1 | RvE1 |
| 17,18-dihydroxy-eicosatetraenoic acid | 17,18-DiHETE |
| 14,15-dihydroxy-eicosatetraenoic acid | 14,15-DiHETE |
| Maresin 1 | Mar-1 |
| Protectin DX | PDX |
| 19,20-epoxy-docosapentaenoic acid | 19(20)-EpDoPE |
| 16,17-epoxy-docosapentaenoic acid | 16(17)-EpDoPE |
| 4-hydroxy-docosahexaenoic acid | 4-HDoHE |
| 17-hydroxy-docosahexaenoic acid | 17-HDoHE |
| 14-hydroxy-docosahexaenoic acid | 14-HDoHE |
| Resolvin D2 | RvD2 |
| Resolvin D1 | RvD1 |
| 19,20-dihydroxy-docosapentaenoic acid | 19,20-DiHDoPE |
| N-(2-hydroxyethyl)-hexadecanamide | PEA |
| Stearoyl Ethanolamide | SEA |
| Oleoyl Ethanolamide | OEA |
| Linoleoyl Ethanolamide | LEA |
| Alpha Linoleoyl Ethanolamide | aLEA |
| Arachidonoyl Ethanolamide | AEA |
| Docosatetraenoyl Ethanolamide | DEA |
| Prostaglandin E2 ethanolamine | PGE2 EA |
| Docosahexaenoyl Ethanolamide | DHEA |
| Progstaglandin F2a ethanolamine | PGF2a EA |
| Progstaglandin D2 ethanolamine | PGD2 EA |
| 15-hydroxy-eicosatetraenate ethanolamine | 15-HETE EA |
| 11,(12)-epoxy-eicosatrienate ethanolamine | 11(12)-EpETre EA |
| 5-hydroxy-12-oxoicosa-6,8,14-trienedioic acid | 20-COOH-LTB4; 12-oxo-10 |
| 20-hydroxy LTB4 | 20-OH-LTB4 |
| Prostaglandin D1 | PGD3 |
| 9,10,13-trihydroxy-11-octadecenoic acid | 9,10,13-TriHOME |
| Prostaglandin D3 | PGD1 |
| 11,12,15-trihydroxyicosa-8,11,13-trienoic acid | 11,12-,15-TriHETrE |
| Prostaglandin J2 | PGJ2 |
| Prostaglandin B2 | PGB2 |
| 6-trans-Leukotriene B4 | 6-trans-LTB4 |
| 11,12-dihydroxy-eicosatetraenoic acid | 11,12-DiHETE |
| 8,9-dihydroxy-eicosatetraenoic acid | 8,9-DiHETE |
| 19,20-dihydroxy-docosapentaenoic acid | 19,20-DiHDPE |
| 12-Oxo-c-LTB3 | LTB3 |
| 16,17-dihydroxy-docosapentaenoic acid | 16,17-DiHDPE |
| 13,14-dihydroxy-docosapentaenoic acid | 13,14-DiHDPE |
| 9S-hydroxy-octadecatrienoic acid | 9-HOTrE |
| 10,11-dihydroxy-docosapentaenoic acid | 10,11-DiHDPE |
| 9-oxo-11-(3-pentyloxiran-2-yl)undec-10-enoic acid | EKODE |
| 13S-hydroxy-octadecatrienoic acid | 13-HOTrE |
| 5S,6R-dihydroxy-eicosatetraenoic acid | 5,6-DiHETE |
| 15-deoxy-δ-12,14-Prostaglandin J2 | 15-deoxy-PGJ2 |
| 7,8-dihydroxy-docosapentaenoic acid | 7,8-DiHDPE |
| 8-hydroxy-eicosapentaenoic acid | 8-HEPE |
| 4,5-diyhdroxy-docosapentaenoic acid | 4,5-DiHDPE |
| 13-keto-octadecadienoic acid | 13-oxo-ODE |
| 15-oxo-eicosatetraenoic acid | 15-oxo-ETE |
| 9-oxo-10,12-octadecadienoic acid | 9-oxo-ODE |
| 8(9)-epoxy-eicosatetraenoic acid | 8(9)-EpETE |
| 15S-hydroxy-eicosatrienoic acid | 15(S)-HETrE |
| 12-oxo-eicosatetraenoic acid | 12-oxo-ETE |
| 19(20)-epoxy-docosapentaenoic acid | 19(20)-EpDPE |
| 16(17)-epoxy-docosapentaenoic acid | 16(17)-EpDPE |
| 13(14)-epoxy-docosapentaenoic acid | 13(14)-EpDPE |
| 5-oxo-eicosatetraenoic acid | 5-oxo-ETE |
| 10(11)-epoxy-docosapentaenoic acid | 10(11)-EpDPE |
| 7(8)-epoxy-docosapentaenoic acid | 7(8)-EpDPE |
| N-8(9)-epoxy-eicosatrienoyl)-ethanolamine | 8(9)-EpETrE alt; 8(9)- |
| | EpETrE-EA |
| 5,6-epoxy-eicosatrienoic acid | 5(6)-EpETrE; 5,6-EET |
| Leukotriene C4 | LTC4 |
| Leukotriene D4 | LTD4 |
| Leukotriene E4 | LTE4 |

TABLE 2
Further Example Oxylipin Information

| Oxylipin Abbreviation | Parent Lipid | Class(es) |
|---|---|---|
| 9(10)-EpO | C18:1n9 | Epox |
| 10-Nitrooleate | C18:1n9 | Nitrolipid |
| 9-Nitrooleate | C18:1n9 | Nitrolipid |
| 9,10-e-DiHO | C18:1n9 | vic-Diol |
| 9(10)-EpOME | C18:2n6 | Epox |
| 12(13)-EpOME | C18:2n6 | Epox |
| 10-Nitrolinoleate | C18:2n6 | Nitrolipid |
| 9-KODE | C18:2n6 | R═O |
| 13-KODE | C18:2n6 | R═O |
| 12(13)Ep-9-KODE | C18:2n6 | R═O, Epox |
| 13-HODE | C18:2n6 | R—OH |
| 9-HODE | C18:2n6 | R—OH |
| 13-HpODE | C18:2n6 | R—OOH |
| 9-HpODE | C18:2n6 | R—OOH |
| 9,12,13-TriHOME | C18:2n6 | Triol |
| 9,10-DiHOME | C18:2n6 | vic-Diol |
| 12,13-DiHOME | C18:2n6 | vic-Diol |
| 9(10)-EpODE | C18:3n3 | Epox |
| 15(16)-EpODE | C18:3n3 | Epox |
| 12(13)-EpODE | C18:3n3 | Epox |
| 9-HOTE | C18:3n3 | R—OH |
| 13-HOTE | C18:3n3 | R—OH |
| 9,10-DiHODE | C18:3n3 | vic-Diol |
| 15,16-DiHODE | C18:3n3 | vic-Diol |
| 12,13-DiHODE | C18:3n3 | vic-Diol |
| PGE1 | C20:3n6 | PG |
| 8,15-DiHETE | C20:4n6 | Diol |
| 5,15-DiHETE | C20:4n6 | Diol |
| LTB4 | C20:4n6 | Diol |
| 8(9)-EpETrE | C20:4n6 | Epox |
| 14(15)-EpETrE | C20:4n6 | Epox |
| 11(12)-EpETrE | C20:4n6 | Epox |
| PGD2 | C20:4n6 | PG |
| 6-keto-PGF1a | C20:4n6 | PG |
| 15-keto-PGE2 | C20:4n6 | PG |
| 15-deoxy PGJ2 | C20:4n6 | PG |
| PGE2 | C20:4n6 | PG |
| PGF2a | C20:4n6 | PG |
| 15-KETE | C20:4n6 | R═O |
| 5-KETE | C20:4n6 | R═O |
| 8-HETE | C20:4n6 | R—OH |
| 9-HETE | C20:4n6 | R—OH |
| 15-HETE | C20:4n6 | R—OH |
| 12-HETE | C20:4n6 | R—OH |
| 11-HETE | C20:4n6 | R—OH |
| 5-HETE | C20:4n6 | R—OH |
| 20-HETE | C20:4n6 | R—OH |
| LxA4 | C20:4n6 | Triol |
| LxB4 | C20:4n6 | Triol |
| TXB2 | C20:4n6 | TX |
| 14,15-DiHETrE | C20:4n6 | vic-Diol |
| 11,12-DiHETrE | C20:4n6 | vic-Diol |
| 8,9-DiHETrE | C20:4n6 | vic-Diol |
| 5,6-DiHETrE | C20:4n6 | vic-Diol |
| RvE2 | C20:5n3 | Diol |
| LTB5 | C20:5n3 | Diol |
| 17(18)-EpETE | C20:5n3 | Epox |
| 14(15)-EpETE | C20:5n3 | Epox |
| 11(12)-EpETE | C20:5n3 | Epox |
| PGE3 | C20:5n3 | PG |
| PGF3a | C20:5n3 | PG |
| 9-HEPE | C20:5n3 | R—OH |
| 5-HEPE | C20:5n3 | R—OH |
| 18-HEPE | C20:5n3 | R—OH |
| 15-HEPE | C20:5n3 | R—OH |
| 12-HEPE | C20:5n3 | R—OH |
| RvE1 | C20:5n3 | Triol |
| 17,18-DiHETE | C20:5n3 | vic-Diol |
| 14,15-DiHETE | C20:5n3 | vic-Diol |
| Mar-1 | C22:6n3 | Diol |
| PDX | C22:6n3 | Diol |
| 19(20)-EpDoPE | C22:6n3 | Epox |
| 16(17)-EpDoPE | C22:6n3 | Epox |
| 4-HDoHE | C22:6n3 | R—OH |
| 17-HDoHE | C22:6n3 | R—OH |
| 14-HDoHE | C22:6n3 | R—OH |
| RvD2 | C22:6n3 | Triol |
| RvD1 | C22:6n3 | Triol |
| 19,20-DiHDoPE | C22:6n3 | vic-Diol |
| PEA | C16:0 | Ethanolamide |
| SEA | C18:0 | Ethanolamide |
| OEA | C18:1n9 | Ethanolamide |
| LEA | C18:2n6 | Ethanolamide |
| aLEA | C18:3n3 | Ethanolamide |
| AEA | C20:4n6 | Ethanolamide |
| DEA | C22:4n6 | Ethanolamide |
| PGE2 EA | C20:4n6 | Ethanolamide |
| DHEA | C22:6n3 | Ethanolamide |
| PGF2a EA | PGF2a | Ethanolamide |
| PGD2 EA | PGD2 | Ethanolamide |
| 15-HETE EA | C20:4n6 | Ethanolamide |
| 11(12)-EpETre EA | C20:4n6 | Ethanolamide |
| 20-COOH-LTB4 | AA | LOX |
| 20-OH-LTB4 | AA | LOX |
| PGD3 | AA | COX2 |
| 9,10,13-TriHOME | LA | LOX |
| PGD1 | AA | COX2 |
| 11,12-,15-TriHETrE | AA | CYP, LOX |
| PGJ2 | AA | COX2 |
| PGB2 | AA | COX2 |
| 6-trans-LTB4 | AA | LOX |
| 11,12-DiHETE | EPA | sEH |
| 8,9-DiHETE | EPA | sEH |
| 19,20-DiHDPE | DHA | sEH |
| LTB3 | AA | LOX |
| 16,17-DiHDPE | DHA | sEH |
| 13,14-DiHDPE | DHA | sEH |
| 9-HOTrE | ALA | LOX |
| 10,11-DiHDPE | DHA | sEH |
| EKODE | AA | oxidation |
| 13-HOTrE | ALA | LOX |
| 5,6-DiHETE | EPA | sEH |
| 15-deoxy-PGJ2 | AA | CYP |
| 7,8-DiHDPE | DHA | sEH |
| 8-HEPE | EPA | LOX |
| 4,5-DiHDPE | DHA | sEH |
| 13-oxo-ODE | LA | ADH |
| 15-oxo-ETE | AA | ADH |
| 9-oxo-ODE | LA | ADH |
| 8(9)-EpETE | EPA | CYP |
| 15(S)-HETrE | DGLA | LOX |
| 12-oxo-ETE | AA | ADH |
| 19(20)-EpDPE | DHA | CYP |
| 16(17)-EpDPE | DHA | CYP |
| 13(14)-EpDPE | DHA | CYP |
| 5-oxo-ETE | AA | ADH |
| 10(11)-EpDPE | DHA | CYP |
| 7(8)-EpDPE | DHA | CYP |
| 8(9)-EpETrE alt | AA | CYP |
| 5(6)-EpETrE | AA | CYP |
| LTC4 | AA | LOX |
| LTD4 | AA | LOX |

Any of the following oxylipins may be used in the methods described herein: 9(10)-EpO, 10-Nitrooleate, 9-Nitrooleate, 9,10-e-DiHO, 9(10)-EpOME, 12(13)-EpOME, 10-Nitrolinoleate, 9-KODE, 13-KODE, 12(13)Ep-9-KODE, 13-HODE, 9-HODE, 13-HpODE, 9-HpODE, 9,12,13-TriHOME, 9,10-DiHOME, 12,13-DiHOME, 9(10)-EpODE, 15(16)-EpODE, 12(13)-EpODE, 9-HOTE, 13-HOTE, 9,10-DiHODE, 15,16-DiHODE, 12,13-DiHODE, PGE1, 8,15-DiHETE, 5,15-DiHETE, LTB4, 8(9)-EpETrE, 14(15)-EpETrE, 11(12)-EpETrE, PGD2, 6-keto-PGF1a, 15-keto-PGE2, 15-deoxy PGJ2, PGE2, PGF2a, 15-KETE, 5-KETE, 8-HETE, 9-HETE, 15-HETE, 12-HETE, 11-HETE, 5-HETE, 20-HETE, LxA4, LxB4, TXB2, 14,15-DiHETrE, 11,12-DiHETrE, 8,9-DiHETrE, 5,6-DiHETrE, RvE2, LTB5, 17(18)-EpETE, 14(15)-EpETE, 11(12)-EpETE, PGE3, PGF3a, 9-HEPE, 5-HEPE, 18-HEPE, 15-HEPE, 12-HEPE, RvE1, 17,18-DiHETE, 14,15-DiHETE, Mar-1, PDX, 19(20)-EpDoPE, 16(17)-EpDoPE, 4-HDoHE, 17-HDoHE, 14-HDoHE, RvD2, RvD1, 19,20-DiHDoPE, PEA, SEA, OEA, LEA, aLEA, AEA, DEA, PGE2 EA, DHEA, PGF2a EA, PGD2 EA, 15-HETE EA, 11(12)-EpETre EA, 20-COOH-LTB4, 20-OH-LTB4, PGD3, 9,10,13-TriHOME, PGD1, 11,12-,15-TriHETrE, PGJ2, PGB2, 6-trans-LTB4, 11,12-DiHETE, 8,9-DiHETE, 19,20-DiHDPE, LTB3, 16,17-DiHDPE, 13,14-iHDPE, 9-HOTrE, 10,11-DiHDPE, EKODE, 13-HOTrE, 5,6-DiHETE, 15-deoxy-PGJ2, 7,8-DiHDPE, 8-HEPE, 4,5-DiHDPE, 13-oxo-ODE, 15-oxo-ETE, 9-oxo-ODE, 8(9)-EpETE, 15(S)-HETrE, 12-oxo-ETE, 19(20)-EpDPE, 16(17)-EpDPE, 13(14)-EpDPE, 5-oxo-ETE, 10(11)-EpDPE, 7(8)-EpDPE, 8(9)-EpETrE, alt 5(6)-EpETrE, LTC4, LTD4, or LTE4. Any of the following oxylipins may be used in the methods described herein: 9(10)-EpO, 10-Nitrooleate, 9-Nitrooleate, 9,10-e-DiHO, 9(10)-EpOME, 12(13)-EpOME, 10-Nitrolinoleate, 9-KODE, 13-KODE, 12(13)Ep-9-KODE, 13-HODE, 9-HODE, 13-HpODE, 9-HpODE, 9,12,13-TriHOME, 9,10-DiHOME, 12,13-DiHOME, 9(10)-EpODE, 15(16)-EpODE, 12(13)-EpODE, 9-HOTE, 13-HOTE, 9,10-DiHODE, 15,16-DiHODE, 12,13-DiHODE, PGE1, 8,15-DiHETE, 5,15-DiHETE, LTB4, 8(9)-EpETrE, 14(15)-EpETrE, 11(12)-EpETrE, PGD2, 6-keto-PGF1a, 15-keto-PGE2, 15-deoxy PGJ2, PGE2, PGF2a, 15-KETE, 5-KETE, 8-HETE, 9-HETE, 15-HETE, 12-HETE, 11-HETE, 5-HETE, 20-HETE, LxA4, LxB4, TXB2, 14,15-DiHETrE, 11,12-DiHETrE, 8,9-DiHETrE, 5,6-DiHETrE, RvE2, LTB5, 17(18)-EpETE, 14(15)-EpETE, 11(12)-EpETE, PGE3, PGF3a, 9-HEPE, 5-HEPE, 18-HEPE, 15-HEPE, 12-HEPE, RvE1, 17,18-DiHETE, 14,15-DiHETE, Mar-1, PDX, 19(20)-EpDoPE, 16(17)-EpDoPE, 4-HDoHE, 17-HDoHE, 14-HDoHE, RvD2, RvD1, 19,20-DiHDoPE, PEA, SEA, OEA, LEA, aLEA, AEA, DEA, PGE2 EA, DHEA, PGF2a EA, PGD2 EA, 15-HETE EA, or 11(12)-EpETre EA. An oxylipin may include 9(10)-EpO. An oxylipin may include 10-Nitrooleate. An oxylipin may include 9-Nitrooleate. An oxylipin may include 9,10-e-DiHO. An oxylipin may include 9(10)-EpOME. An oxylipin may include 12(13)-EpOME. An oxylipin may include 10-Nitrolinoleate. An oxylipin may include 9-KODE. An oxylipin may include 13-KODE. An oxylipin may include 12(13)Ep-9-KODE. An oxylipin may include 13-HODE. An oxylipin may include 9-HODE. An oxylipin may include 13-HpODE. An oxylipin may include 9-HpODE. An oxylipin may include 9,12,13-TriHOME. An oxylipin may include 9,10-DiHOME. An oxylipin may include 12,13-DiHOME. An oxylipin may include 9(10)-EpODE. An oxylipin may include 15(16)-EpODE. An oxylipin may include 12(13)-EpODE. An oxylipin may include 9-HOTE. An oxylipin may include 13-HOTE. An oxylipin may include 9,10-DiHODE. An oxylipin may include 15,16-DiHODE. An oxylipin may include 12,13-DiHODE. An oxylipin may include PGE1. An oxylipin may include 8,15-DiHETE. An oxylipin may include 5,15-DiHETE. An oxylipin may include LTB4. An oxylipin may include 8(9)-EpETrE. An oxylipin may include 14(15)-EpETrE. An oxylipin may include 11(12)-EpETrE. An oxylipin may include PGD2. An oxylipin may include 6-keto-PGF1a. An oxylipin may include 15-keto-PGE2. An oxylipin may include 15-deoxy PGJ2. An oxylipin may include PGE2. An oxylipin may include PGF2a. An oxylipin may include 15-KETE. An oxylipin may include 5-KETE. An oxylipin may include 8-HETE. An oxylipin may include 9-HETE. An oxylipin may include 15-HETE. An oxylipin may include 12-HETE. An oxylipin may include 11-HETE. An oxylipin may include 5-HETE. An oxylipin may include 20-HETE. An oxylipin may include LxA4. An oxylipin may include LxB4. An oxylipin may include TXB2. An oxylipin may include 14,15-DiHETrE. An oxylipin may include 11,12-DiHETrE. An oxylipin may include 8,9-DiHETrE. An oxylipin may include 5,6-DiHETrE. An oxylipin may include RvE2. An oxylipin may include LTB5. An oxylipin may include 17(18)-EpETE. An oxylipin may include 14(15)-EpETE. An oxylipin may include 11(12)-EpETE. An oxylipin may include PGE3. An oxylipin may include PGF3a. An oxylipin may include 9-HEPE. An oxylipin may include 5-HEPE. An oxylipin may include 18-HEPE. An oxylipin may include 15-HEPE. An oxylipin may include 12-HEPE. An oxylipin may include RvE1. An oxylipin may include 17,18-DiHETE. An oxylipin may include 14,15-DiHETE. An oxylipin may include Mar-1. An oxylipin may include PDX. An oxylipin may include 19(20)-EpDoPE. An oxylipin may include 16(17)-EpDoPE. An oxylipin may include 4-HDoHE. An oxylipin may include 17-HDoHE. An oxylipin may include 14-HDoHE. An oxylipin may include RvD2. An oxylipin may include RvD1. An oxylipin may include 19,20-DiHDoPE. An oxylipin may include PEA. An oxylipin may include SEA. An oxylipin may include OEA. An oxylipin may include LEA. An oxylipin may include aLEA. An oxylipin may include AEA. An oxylipin may include DEA. An oxylipin may include PGE2 EA. An oxylipin may include DHEA. An oxylipin may include PGF2a EA. An oxylipin may include PGD2 EA. An oxylipin may include 15-HETE EA. An oxylipin may include 11(12)-EpETre EA. An oxylipin may include F2-IsoPs. An oxylipin may include 20-COOH-LTB4. An oxylipin may include 20-OH-LTB4. An oxylipin may include PGD3. An oxylipin may include 9,10,13-TriHOME. An oxylipin may include PGD1. An oxylipin may include 11,12-,15-TriHETrE. An oxylipin may include PGJ2. An oxylipin may include PGB2. An oxylipin may include 6-trans-LTB4. An oxylipin may include 11,12-DiHETE. An oxylipin may include 8,9-DiHETE. An oxylipin may include 19,20-DiHDPE. An oxylipin may include LTB3. An oxylipin may include 16,17-DiHDPE. An oxylipin may include 13,14-DiHDPE. An oxylipin may include 9-HOTrE. An oxylipin may include 10,11-DiHDPE. An oxylipin may include EKODE. An oxylipin may include 13-HOTrE. An oxylipin may include 5,6-DiHETE. An oxylipin may include 15-deoxy-PGJ2. An oxylipin may include 7,8-DiHDPE. An oxylipin may include 8-HEPE. An oxylipin may include 4,5-DiHDPE. An oxylipin may include 13-oxo-ODE. An oxylipin may include 15-oxo-ETE. An oxylipin may include 9-oxo-ODE. An oxylipin may include 8(9)-EpETE. An oxylipin may include 15(S)-HETrE. An oxylipin may include 12-oxo-ETE. An oxylipin may include 19(20)-EpDPE. An oxylipin may include 16(17)-EpDPE. An oxylipin may include 13(14)-EpDPE. An oxylipin may include 5-oxo-ETE. An oxylipin may include 10(11)-EpDPE. An oxylipin may include 7(8)-EpDPE. An oxylipin may include 8(9)-EpETrE alt. An oxylipin may include 5(6)-EpETrE. An oxylipin may include LTC4. An oxylipin may include LTD4. An oxylipin may include LTE4. In some embodiments, any of the following oxylipins are used: 9(10)-EpO, 10-Nitrooleate, 9-Nitrooleate, 9,10-e-DiHO, 9(10)-EpOME, 12(13)-EpOME, 10-Nitrolinoleate, 9-KODE, 13-KODE, 12(13)Ep-9-KODE, 9-HODE, 13-HpODE, 9-HpODE, 9,12,13-TriHOME, 9,10-DiHOME, 12,13-DiHOME, 9(10)-EpODE, 15(16)-EpODE, 12(13)-EpODE, 9-HOTE, 13-HOTE, 9,10-DiHODE, 15,16-DiHODE, 12,13-DiHODE, PGE1, 8,15-DiHETE, 5,15-DiHETE, LTB4, 8(9)-EpETrE, 14(15)-EpETrE, 11(12)-EpETrE, PGD2, 6-keto-PGF1a, 15-keto-PGE2, 15-deoxy PGJ2, PGE2, PGF2a, 15-KETE, 5-KETE, 8-HETE, 9-HETE, 11-HETE, 5-HETE, 20-HETE, LxA4, LxB4, TXB2, 14,15-DiHETrE, 11,12-DiHETrE, 8,9-DiHETrE, 5,6-DiHETrE, RvE2, LTB5, 17(18)-EpETE, 14(15)-EpETE, 11(12)-EpETE, PGE3, PGF3a, 9-HEPE, 5-HEPE, 18-HEPE, 15-HEPE, 12-HEPE, 17,18-DiHETE, 14,15-DiHETE, Mar-1, PDX, 19(20)-EpDoPE, 16(17)-EpDoPE, 4-HDoHE, 17-HDoHE, 14-HDoHE, RvD2, 19,20-DiHDoPE, PEA, SEA, OEA, LEA, aLEA, AEA, DEA, PGE2 EA, DHEA, PGF2a EA, PGD2 EA, 15-HETE EA, or 11(12)-EpETre EA. In some embodiments, any of the following oxylipins are used: 20-COOH-LTB4, 20-OH-LTB4, PGD3, 9,10,13-TriHOME, PGD1, 11,12-,15-TriHETrE, PGJ2, PGB2, 6-trans-LTB4, 11,12-DiHETE, 8,9-DiHETE, 19,20-DiHDPE, LTB3, 16,17-DiHDPE, 13,14-iHDPE, 9-HOTrE, 10,11-DiHDPE, EKODE, 13-HOTrE, 5,6-DiHETE, 15-deoxy-PGJ2, 7,8-DiHDPE, 8-HEPE, 4,5-DiHDPE, 13-oxo-ODE, 15-oxo-ETE, 9-oxo-ODE, 8(9)-EpETE, 15(S)-HETrE, 12-oxo-ETE, 19(20)-EpDPE, 16(17)-EpDPE, 13(14)-EpDPE, 5-oxo-ETE, 10(11)-EpDPE, 7(8)-EpDPE, 8(9)-EpETrE, alt 5(6)-EpETrE, LTC4, LTD4, or LTE4. In some embodiments, any of the following oxylipins are used: 5-HEPE, 12-HETE, 9-HETE, or 13-HODE. Any number or combination of any of the aforementioned oxylipins may be used in a method described herein.

In some embodiments, the oxylipin comprises an epoxide oxylipin. In some embodiments, the oxylipin comprises a hydroxyl oxylipin. In some embodiments, the oxylipin comprises a keto oxylipin. In some embodiments, the oxylipin is arachidonic acid (AA)-derived. In some embodiments, the oxylipin is eicosapentaenoic acid (EPA)-derived. In some embodiments, oxylipin is docosahexaenoic acid (DHA)-derived. In some embodiments, the oxylipin is linoleic acid (LA)-derived. In some embodiments, the oxylipin is alpha-linoleic acid (ALA)-derived. In some embodiments, the oxylipin is dihomo-γ-linolenic acid (DGLA)-derived.

In some embodiments, the oxylipin is in a free fatty acid form. For example, a non-esterified or free fatty acid form of an oxylipin may be useful as a biomarker. In some embodiments, the oxylipin comprises a free fatty acid. In some embodiments, the oxylipin comprises a non-esterified fatty acid. In some embodiments, the oxylipin is non-esterified.

In some embodiments, the oxylipin is in an esterified form. For example, an esterified oxylipin may be useful as a biomarker. In some embodiments, the oxylipin is esterified. In some embodiments, the oxylipin comprises an ester.

In some embodiments, the oxylipin is endogenous to a subject. In some embodiments, the oxylipin is derived from an exogenous compound administered to a subject. For example, the oxylipin may be derived from a parent lipid (e.g. C18:1n9, C18:2n6, C18:3n3, C20:3n6, C20:4n6, C20:5n3, C22:6n3, C16:0, C18:0, C18:1n9, C18:2n6, C18:3n3, C20:4n6, C22:4n6, C22:6n3, PGF2a, or PGD2). The parent lipid may be labeled prior to administration. As such, an oxylipin derived from a labeled parent lipid may include the same label as the parent lipid. An example of a label is an isotopic label such as ¹³C or ²H. Any parent in Table 2 may be labeled.

The labeled parent may include labeled linoleic acid (e.g. ¹³C linoleic acid). Examples of labeled oxylipins that may be derived from a labeled linoleic acid may include 9(10)-EpOME, 12(13)-EpOME, 10-Nitrolinoleate, 9-KODE, 13-KODE, 12(13)Ep-9-KODE, 13-HODE, 9-HODE, 13-HpOD, 9-HpODE, 9,12,13-TriHOME, 9,10-DiHOME, or 12,13-DiHOME. A labeled oxylipins derived from a labeled linoleic acid may include 9(10)-EpOME, 12(13)-EpOME, 10-Nitrolinoleate, 9-KODE, 13-KODE, 12(13)Ep-9-KODE, 9-HODE, 13-HpODE, 9-HpODE, 9,12,13-TriHOME, 9,10-DiHOME, or 12,13-DiHOME. Additional examples may be found in Table 2.

The labeled parent may include labeled alpha-linoleic acid (e.g. ¹³C alpha-linoleic acid). Examples of labeled oxylipins that may be derived from a labeled alpha-linoleic acid may include 9(10)-EpODE, 15(16)-EpODE, 12(13)-EpODE, 9-HOTE, 13-HOTE, 9,10-DiHODE, 15,16-DiHODE, or 12,13-DiHODE. Additional examples may be found in Table 2.

Some embodiments include obtaining an oxylipin measurement. Said obtaining may include receiving. For example, some embodiments include receiving an oxylipin measurement. Said obtaining may include measuring or assaying. For example, some embodiments include measuring an oxylipin. Some embodiments include assaying an oxylipin. A method may include obtaining a measurement of an oxylipin in Table 1. A method may include obtaining a measurement of an oxylipin selected from a grouping within Table 2. The grouping may include a parent lipid (e.g. C18:1n9, C18:2n6, C18:3n3, C20:3n6, C20:4n6, C20:5n3, C22:6n3, C16:0, C18:0, C18:1n9, C18:2n6, C18:3n3, C20:4n6, C22:4n6, C22:6n3, PGF2a, or PGD2). The grouping may include a class (e.g. Epox, Nitrolipid, vic-Diol, R═O, R—OH, R—OOH, Triol, PG, Diol, TX, or Ethanolamide). Where one oxylipin measurement is described, it is contemplated that multiple oxylipin measurements may be used or obtained, or vice versa.

Some embodiments include obtaining oxylipin measurements. In some embodiments, the oxylipin measurements include 2, 3, 4, 5, 6, 7, 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, or 90 different oxylipins, or a range defined by any two of the aforementioned numbers of oxylipins. Some embodiments include obtaining oxylipin measurements. In some embodiments, the oxylipin measurements include 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, or 132 different oxylipins, or a range defined by any two of the aforementioned numbers of oxylipins. Some methods include the use of about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, about 35, about 36, about 37, about 38, about 39, about 40, about 41, about 42, about 43, about 44, about 45, about 46, about 47, about 48, about 49, about 50, about 51, about 52, about 53, about 54, about 55, about 56, about 57, about 58, about 59, about 60, about 61, about 62, about 63, about 64, about 65, about 66, about 67, about 68, about 69, about 70, about 71, about 72, about 73, about 74, about 75, about 76, about 77, about 78, about 79, about 80, about 81, about 82, about 83, about 84, about 85, about 86, about 87, about 88, about 89, about 90, about 91, about 92, about 93, about 94, about 95, about 96, about 97, about 98, about 99, about 100, about 101, about 102, about 103, about 104, about 105, about 106, about 107, about 108, about 109, about 110, about 111, about 112, about 113, about 114, about 115, about 116, about 117, about 118, about 119, about 120, about 121, about 122, about 123, about 124, about 125, about 126, about 127, about 128, about 129, about 130, about 131, or about 132 oxylipins or oxylipin measurements. Some embodiments include obtaining at least 2 different oxylipin measurements, at least 3 different oxylipin measurements, at least 4 different oxylipin measurements, at least 5 different oxylipin measurements, at least 6 different oxylipin measurements, at least 7 different oxylipin measurements, at least 8 different oxylipin measurements, at least 9 different oxylipin measurements, at least 10 different oxylipin measurements, at least 11 different oxylipin measurements, at least 12 different oxylipin measurements, at least 13 different oxylipin measurements, at least 14 different oxylipin measurements, at least 15 different oxylipin measurements, at least 16 different oxylipin measurements, at least 17 different oxylipin measurements, at least 18 different oxylipin measurements, at least 19 different oxylipin measurements, at least 20 different oxylipin measurements, at least 21 different oxylipin measurements, at least 22 different oxylipin measurements, at least 23 different oxylipin measurements, at least 24 different oxylipin measurements, at least 25 different oxylipin measurements, at least 30 different oxylipin measurements, at least 35 different oxylipin measurements, at least 40 different oxylipin measurements, at least 45 different oxylipin measurements, at least 50 different oxylipin measurements, at least 55 different oxylipin measurements, at least 60 different oxylipin measurements, at least 65 different oxylipin measurements, at least 70 different oxylipin measurements, at least 75 different oxylipin measurements, at least 80 different oxylipin measurements, at least 85 different oxylipin measurements, or at least or 90 different oxylipin measurements. Some embodiments include obtaining at least 95 different oxylipin measurements, at least 100 different oxylipin measurements, at least 105 different oxylipin measurements, at least 110 different oxylipin measurements, at least 115 different oxylipin measurements, at least 120 different oxylipin measurements, at least 125 different oxylipin measurements, at least 130 different oxylipin measurements, or at least 132 different oxylipin measurements. Some embodiments include obtaining no more than 2 different oxylipin measurements, no more than 3 different oxylipin measurements, no more than 4 different oxylipin measurements, no more than 5 different oxylipin measurements, no more than 6 different oxylipin measurements, no more than 7 different oxylipin measurements, no more than 8 different oxylipin measurements, no more than 9 different oxylipin measurements, no more than 10 different oxylipin measurements, no more than 11 different oxylipin measurements, no more than 12 different oxylipin measurements, no more than 13 different oxylipin measurements, no more than 14 different oxylipin measurements, no more than 15 different oxylipin measurements, no more than 16 different oxylipin measurements, no more than 17 different oxylipin measurements, no more than 18 different oxylipin measurements, no more than 19 different oxylipin measurements, no more than 20 different oxylipin measurements, no more than 21 different oxylipin measurements, no more than 22 different oxylipin measurements, no more than 23 different oxylipin measurements, no more than 24 different oxylipin measurements, no more than 25 different oxylipin measurements, no more than 30 different oxylipin measurements, no more than 35 different oxylipin measurements, no more than 40 different oxylipin measurements, no more than 45 different oxylipin measurements, no more than 50 different oxylipin measurements, no more than 55 different oxylipin measurements, no more than 60 different oxylipin measurements, no more than 65 different oxylipin measurements, no more than 70 different oxylipin measurements, no more than 75 different oxylipin measurements, no more than 80 different oxylipin measurements, no more than 85 different oxylipin measurements, or no more than or 90 different oxylipin measurements. Some embodiments include obtaining no more than 95 different oxylipin measurements, no more than 100 different oxylipin measurements, no more than 105 different oxylipin measurements, no more than 110 different oxylipin measurements, no more than 115 different oxylipin measurements, no more than 120 different oxylipin measurements, no more than 125 different oxylipin measurements, no more than 130 different oxylipin measurements, or no more than 132 different oxylipin measurements. The different oxylipin measurements may be measurements of oxylipins in Table 1, for example any combination or number of oxylipins in Table 1. The different oxylipin measurements may be measurements of oxylipins in a grouping within Table 2, for example any combination or number of groupings in Table 2. The grouping may include a parent lipid (e.g. C18:1n9, C18:2n6, C18:3n3, C20:3n6, C20:4n6, C20:5n3, C22:6n3, C16:0, C18:0, C18:1n9, C18:2n6, C18:3n3, C20:4n6, C22:4n6, C22:6n3, PGF2a, or PGD2). The grouping may include a class (e.g. Epox, Nitrolipid, vic-Diol, R═O, R—OH, R—OOH, Triol, PG, Diol, TX, or Ethanolamide). Some embodiments include obtaining measurements of some of the oxylipins in Table 1. Some embodiments include obtaining measurements of all of the oxylipins in Table 1. The measurements may exclude any oxylipin in Table 1. For example, the measurements may exclude 13-HODE, 15-HETE, 12-HETE, RvE1, RvD1, LTB3, PDX, or Mar-1. 13-HODE may be excluded. 15-HETE may be excluded. 12-HETE may be excluded. RvE1 may be excluded. RvD1 may be excluded. LTB3 may be excluded. PDX may be excluded. Mar-1 may be excluded. Some embodiments comprise measurements of an oxylipin or oxylipins other than any of the oxylipins disclosed herein (e.g. other than 13-HODE, 15-HETE, 12-HETE, RvE1, RvD1, LTB3, PDX, or Mar-1). Some embodiments include a biomarkers or biomarkers that include an oxylipin or group of oxylipins other than any of the oxylipins disclosed herein (e.g. other than 13-HODE, 15-HETE, 12-HETE, RvE1, RvD1, LTB3, PDX, or Mar-1). A panel of oxylipin measurements may be used in a method described herein.

Disclosed herein, in some embodiments, are oxylipin measurements. In some embodiments, the oxylipin measurement is elevated or is decreased in the subject, relative to a control or baseline oxylipin measurement. In some embodiments, the oxylipin measurement varies from a control oxylipin measurement. The control oxylipin measurement may be a threshold or a mean of a general patient population for oxylipin. In some embodiments, the oxylipin measurement varies from a threshold. In some embodiments, the oxylipin measurement varies from a mean of a general patient population. In some embodiments, the oxylipin measurement varies by at least 1 σ from a mean of the general patient population. In some embodiments, the oxylipin measurement is greater than the control oxylipin measurement, is greater than the threshold, or is greater than the mean of the general patient population for the oxylipin measurement. In some embodiments, the oxylipin measurement is less than the control oxylipin measurement, is less than the threshold, or is less than the mean of the general patient population for the oxylipin measurement. In some embodiments, the oxylipin measurement is greater than a baseline oxylipin measurement. In some embodiments, the oxylipin measurement is less than a baseline oxylipin measurement.

The baseline measurement may be obtained before administration of a HSD17B13 inhibitor. The baseline measurement may be obtained after administration of a HSD17B13 inhibitor. The baseline measurement may be obtained before administration of a HSD17B13 inhibitor. The baseline measurement may be obtained before administration of a first dose of a HSD17B13 inhibitor.

An increase in an oxylipin measurement (or any number or combination of the oxylipins disclosed herein) may be useful as a biomarker. In some embodiments, the increase includes esterified oxylipins. In some embodiments, the increase includes non-esterified oxylipins. A decrease in an oxylipin measurement (or any number or combination of the oxylipins disclosed herein) may be useful as a biomarker. In some embodiments, the decrease includes esterified oxylipins. In some embodiments, the decrease includes non-esterified oxylipins.

### Methods of Treatment

Disclosed herein, in some embodiments, are treatment methods. Some embodiments include obtaining or receiving an oxylipin measurement in a biological sample of a subject. Some embodiments include administering or discontinuing administration of a 17β-hydroxysteroid dehydrogenase type 13 (HSD17B13) inhibitor to the subject. The administration may be based on the oxylipin measurement. Some embodiments include administering the HSD17B13 inhibitor.

In some embodiments, the subject has already received an earlier HSD17B13 inhibitor administration. Some embodiments further include identifying an effective dose of the HSD17B13 inhibitor based on the oxylipin measurement. Some embodiments further include identifying a dose of the HSD17B13 inhibitor based on the oxylipin measurement that reproduces a HSD17B13 phenotype in the subject. In some embodiments, administering the HSD17B13 inhibitor to the subject comprises adjusting a dose of the HSD17B13 inhibitor. Some embodiments include discontinuing administration of the HSD17B13 inhibitor. In some embodiments, administering the HSD17B13 inhibitor to the subject comprises modifying a dose level of the HSD17B13 inhibitor. Some embodiments include obtaining or receiving an oxylipin measurement in a biological sample of a subject; and modifying a dose of a HSD17B13 inhibitor based on the oxylipin measurement.

Provided herein, in some embodiments, are methods for treating a patient. Some embodiments include obtaining one or more biomarker measurements such as an oxylipin measurement. Some embodiments include receiving one or more biomarker measurements. In some embodiments, the biomarker measurements are obtained in a sample from a patient. Some embodiments include obtaining one or more biomarker measurements in at least one sample from a patient. Some embodiments include receiving one or more biomarker measurements in at least one sample from a patient.

Disclosed herein, in some embodiments, are methods for treating a patient. Some embodiments include obtaining or receiving one or more biomarker measurements in at least one sample from a patient, the biomarkers comprising oxylipins such as those in Table 1. Some embodiments include administering a 17β-Hydroxysteroid dehydrogenase type 13 (HSD17B13) inhibitor based on the of the one or more biomarker measurements. The HSD17B13 inhibitor may be any HSD17B13 inhibitor described herein. Disclosed herein, in some embodiments, are methods for treating a patient. Disclosed herein are methods for treating a patient, comprising: obtaining or receiving one or more oxylipin measurements in at least one sample from a patient; and administering a 17β-Hydroxysteroid dehydrogenase type 13 (HSD17B13) inhibitor based on the of the one or more biomarker measurements.

Some embodiments include comparing the one or more biomarker measurements such as an oxylipin measurement to a control measurement or threshold for each biomarker. Some embodiments include comparing the one or more biomarker measurements to a control measurement. Some embodiments include comparing the one or more biomarker measurements to a threshold for each biomarker. In some embodiments, the control measurement is obtained from a patient population such as a general patient population.

Disclosed herein, in some embodiments, are methods for treating a patient based on obtained or received biomarker measurements such as oxylipin measurements. Some embodiments include identifying, based on the obtained or received measurements, the patient as likely to benefit from a treatment with the HSD17B13 inhibitor or as not likely to benefit from the treatment. Some embodiments include, for each biomarker measurement, determining a variance of the biomarker measurement from the control measurement, or determining whether the biomarker measurement exceeds the threshold. Some embodiments include, for each biomarker measurement, determining a variance of the biomarker measurement from the control measurement. Some embodiments include, for each biomarker measurement, determining whether the biomarker measurement exceeds the threshold.

Disclosed herein, in some embodiments, are methods for treating a patient based on obtained or received biomarker measurements such as oxylipin measurements. Some embodiments include, for each biomarker measurement, determining whether the biomarker measurement is predictive of the patient being likely to benefit from the treatment with the HSD17B13 inhibitor based on the variance of the biomarker measurement from the control measurement, or based on whether the biomarker measurement exceeds the threshold. Some embodiments include, for each biomarker measurement, determining whether the biomarker measurement is predictive of the patient being likely to benefit from the treatment with the HSD17B13 inhibitor based on the variance of the biomarker measurement from the control measurement. Some embodiments include, for each biomarker measurement, determining whether the biomarker measurement is predictive of the patient being likely to benefit from the treatment with the HSD17B13 inhibitor based on whether the biomarker measurement exceeds the threshold.

Disclosed herein, in some embodiments, are methods for treating a patient based on obtained or received biomarker measurements such as oxylipin measurements. In some embodiments, a biomarker measurement is predictive of the patient being likely to benefit from the treatment with the HSD17B13 inhibitor. In some embodiments, a biomarker measurement is predictive of the patient being likely to benefit from the treatment with the HSD17B13 inhibitor when the variance is at least 1 σ from a mean of the patient population (e.g. general patient population).

Disclosed herein, in some embodiments, are methods for treating a patient based on obtained or received biomarker measurements such as oxylipin measurements. In some embodiments, one or more biomarkers in Table 1 are predictive of the patient being likely to benefit from the treatment with the HSD17B13 inhibitor when they are greater in the patient than the control measurement or threshold.

Disclosed herein, in some embodiments, are methods for treating a patient based on obtained or received biomarker measurements such as oxylipin measurements. Some embodiments include determining a number of biomarker measurements that are predictive of the patient being likely to benefit from the treatment with the HSD17B13 inhibitor. Some embodiments include identifying the patient as likely to benefit from the treatment with the HSD17B13 inhibitor when the number of biomarker measurements that are predictive is at least 1, at least 2, at least 3, at least 4, at least 5, at least 6, at least 7, at least 8, at least 9, or at least 10, or as not likely to benefit from the treatment when the number of biomarker measurements that are predictive is less than 1, less than 2, less than 3, less than 4, less than 5, less than 6, less than 7, less than 8, less than 9, or less than 10.

Disclosed herein, in some embodiments, are methods for treating a patient based on obtained or received biomarker measurements such as oxylipin measurements. Some embodiments include identifying a cumulative biomarker panel deviation of the patient from a cumulative biomarker mean of the patient population. Some embodiments include identifying the patient as likely to benefit from the treatment with the HSD17B13 inhibitor when the cumulative biomarker panel of the patient deviates sufficiently from the cumulative mean of the patient population, and identifying the patient as unlikely to benefit from the treatment with the HSD17B13 inhibitor when the cumulative biomarker panel deviation of the patient does not deviate sufficiently from the cumulative mean of the patient population. In some embodiments, the deviation is sufficient if it is at least 1 σ from a mean of the patient population. Some embodiments include identifying the patient as likely to benefit from the treatment with the HSD17B13 inhibitor.

Some embodiments relate to a cumulative biomarker panel deviation, or a cumulative biomarker score. The cumulative biomarker panel deviation may be determined through summation of the deviation from the mean. The cumulative biomarker panel deviation may include rank of a number of analytes as deviating from the mean. The cumulative biomarker score may be determined through summation of the deviation from the mean. The cumulative biomarker score may include rank of a number of analytes as deviating from the mean. Some embodiments include a calculation such as: cumulative biomarker score=Sum(absolute value of beta). Some embodiments include: cumulative biomarker score=number of analytes that deviate 1 sigma from mean/total analytes. Some embodiments include a calculation such as: cumulative biomarker score=Sum(absolute value of beta). Some embodiments include: cumulative biomarker score=number of analytes that deviate 1 sigma from mean/total analytes. In some embodiments, the cumulative biomarker score is used to determine whether to treat the patient. In some embodiments, the cumulative biomarker score is generated using the biomarker measurements. Some embodiments include applying a mathematical algorithm to the obtained biomarker levels to obtain the cumulative biomarker score. In some embodiments, the trained algorithm is or has been generated using regression modeling or machine learning on a training data set.

Some embodiments of the methods disclosed herein include providing an HSD17B13 inhibitor to a subject or patient, for example based on whether the patient is identified as likely to benefit from the treatment with the HSD17B13 inhibitor, or based on whether the patient is identified as at risk of having a liver disease as described herein. Some embodiments include administering an HSD17B13 inhibitor to a subject or patient. Some embodiments include providing a liver disease treatment or a composition described herein to a subject or patient in need thereof. Some embodiments include administering a liver disease treatment or a composition described herein to a subject or patient in need thereof. Some embodiments relate to a method comprising administering an HSD17B13 inhibitor to a subject having the liver disease. In some embodiments, the administration includes is by injection. In some embodiments, the administration decreases HSD17B13 activity or expression in the patient. In some embodiments, the composition decreases HSD17B13 activity or expression the patient or subject. In some embodiments, the administration treats the disorder in the patient. In some embodiments, the composition treats the patient or subject. For example, the patient may have a liver disease that is treated by the administration of the HSD17B13 inhibitor.

Some embodiments of the methods disclosed herein include treatment of a liver disease with an HSD17B13 inhibitor. In some embodiments, the liver disease comprises liver inflammation, liver fibrosis, cholestasis, a gall bladder disease, a biliary tree disease, alcoholic liver disease, or non-alcoholic steatohepatitis. In some embodiments, the liver disease comprises liver inflammation. In some embodiments, the liver disease comprises liver fibrosis. In some embodiments, the liver disease comprises cholestasis. In some embodiments, the liver disease comprises a gall bladder disease. In some embodiments, the liver disease comprises a biliary tree disease. In some embodiments, the liver disease comprises alcoholic liver disease, or non-alcoholic steatohepatitis. In some embodiments, the liver disease comprises alcoholic liver disease. In some embodiments, the liver disease comprises non-alcoholic steatohepatitis. In some embodiments, the liver disease is related to a liver disease risk allele of HSD17B13. In some embodiments, the liver disease is related to a liver disease risk allele of PNPLA3.

### Patient Selection for HSDI7B13 Inhibitor Treatments

Provided herein, in some embodiments, are methods for identifying a patient likely to benefit from treatment with an HSD17B13 inhibitor. Some embodiments include obtaining one or more biomarker measurements such as an oxylipin measurement. Some embodiments include receiving one or more biomarker measurements. Some embodiments further include identifying the subject as a candidate for HSD17B13 inhibitor treatment based on the oxylipin measurement. In some embodiments, the biomarker measurements are obtained in a sample from a patient. Some embodiments include obtaining one or more biomarker measurements in at least one sample from a patient. Some embodiments include receiving one or more biomarker measurements in at least one sample from a patient.

Disclosed herein, in some embodiments, are methods for identifying a patient likely to benefit from treatment with an HSD17B13 inhibitor. Some embodiments include obtaining or receiving one or more biomarker measurements in at least one sample from a patient, the biomarkers comprising oxylipins such as those in Table 1.

Disclosed herein, in some embodiments, are methods for identifying a patient likely to benefit from treatment with an HSD17B13 inhibitor. Some embodiments include identifying, based on the obtained or received measurements, the patient as likely to benefit from a treatment with an HSD17B13 inhibitor or as not likely to benefit from the treatment. The HSD17B13 inhibitor may be any HSD17B13 inhibitor described herein. Some embodiments include identifying the patient as likely to benefit from the treatment with the HSD17B13 inhibitor. Some embodiments include providing the treatment with the HSD17B13 inhibitor to the patient.

Provided herein, in some embodiments, are methods for identifying a patient likely to benefit from treatment with an HSD17B13 inhibitor, comprising: obtaining or receiving measurements of one or more biomarkers in at least one sample from a patient, the biomarkers comprising oxylipins such as those in Table 1; and identifying, based on the obtained or received measurements, the patient as likely to benefit from a treatment with an HSD17B13 inhibitor or as not likely to benefit from the treatment.

Disclosed herein, in some embodiments, are methods for identifying a patient likely to benefit from treatment with an HSD17B13 inhibitor. Some embodiments include comparing the one or more biomarker measurements such as oxylipin measurements to a control measurement or threshold for each biomarker. Some embodiments include comparing the one or more biomarker measurements to a control measurement. Some embodiments include comparing the one or more biomarker measurements to a threshold for each biomarker. In some embodiments, the control measurement is obtained from a patient population such as a general patient population.

Disclosed herein, in some embodiments, are methods for identifying a patient likely to benefit from treatment with an HSD17B13 inhibitor based on obtained or received biomarker measurements such as oxylipin measurements. Some embodiments include, for each biomarker measurement, determining a variance of the biomarker measurement from the control measurement, or determining whether the biomarker measurement exceeds the threshold. Some embodiments include, for each biomarker measurement, determining a variance of the biomarker measurement from the control measurement. Some embodiments include, for each biomarker measurement, determining whether the biomarker measurement exceeds the threshold.

Disclosed herein, in some embodiments, are methods for identifying a patient likely to benefit from treatment with an HSD17B13 inhibitor based on obtained or received biomarker measurements such as oxylipin measurements. Some embodiments include, for each biomarker measurement, determining whether the biomarker measurement is predictive of the patient being likely to benefit from the treatment with the HSD17B13 inhibitor based on the variance of the biomarker measurement from the control measurement, or based on whether the biomarker measurement exceeds the threshold. Some embodiments include, for each biomarker measurement, determining whether the biomarker measurement is predictive of the patient being likely to benefit from the treatment with the HSD17B13 inhibitor based on the variance of the biomarker measurement from the control measurement. Some embodiments include, for each biomarker measurement, determining whether the biomarker measurement is predictive of the patient being likely to benefit from the treatment with the HSD17B13 inhibitor based on whether the biomarker measurement exceeds the threshold.

Disclosed herein, in some embodiments, are methods for identifying a patient likely to benefit from treatment with an HSD17B13 inhibitor based on obtained or received biomarker measurements such as oxylipin measurements. In some embodiments, a biomarker measurement is predictive of the patient being likely to benefit from the treatment with the HSD17B13 inhibitor. In some embodiments, a biomarker measurement is predictive of the patient being likely to benefit from the treatment with the HSD17B13 inhibitor when the variance is at least 1 σ from a mean of the patient population (e.g. general patient population).

Disclosed herein, in some embodiments, are methods for identifying a patient likely to benefit from treatment with an HSD17B13 inhibitor based on obtained or received biomarker measurements such as oxylipin measurements. Some embodiments include determining a number of biomarker measurements that are predictive of the patient being likely to benefit from the treatment with the HSD17B13 inhibitor. Some embodiments include identifying the patient as likely to benefit from the treatment with the HSD17B13 inhibitor when the number of biomarker measurements that are predictive is at least 1, at least 2, at least 3, at least 4, at least 5, at least 6, at least 7, at least 8, at least 9, or at least 10, or as not likely to benefit from the treatment when the number of biomarker measurements that are predictive is less than 1, less than 2, less than 3, less than 4, less than 5, less than 6, less than 7, less than 8, less than 9, or less than 10.

Disclosed herein, in some embodiments, are methods for identifying a patient likely to benefit from treatment with an HSD17B13 inhibitor based on obtained or received biomarker measurements such as oxylipin measurements. Some embodiments include identifying a cumulative biomarker panel deviation of the patient from a cumulative mean of the patient population. Some embodiments include identifying the patient as likely to benefit from the treatment with the HSD17B13 inhibitor when the cumulative biomarker panel deviation of the patient deviates sufficiently from the cumulative mean of the patient population, and identifying the patient as unlikely to benefit from the treatment with the HSD17B13 inhibitor when the cumulative biomarker panel deviation of the patient does not deviate sufficiently from the cumulative mean of the patient population. In some embodiments, the deviation is sufficient if it is at least 1 σ from a mean of the patient population.

Some embodiments relate to a cumulative biomarker panel deviation, or a cumulative biomarker score. The cumulative biomarker panel deviation may be determined through summation of the deviation from the mean. The cumulative biomarker panel deviation may include rank of a number of analytes as deviating from the mean. The cumulative biomarker score may be determined through summation of the deviation from the mean. The cumulative biomarker score may include rank of a number of analytes as deviating from the mean. Some embodiments include a calculation such as: cumulative biomarker score=Sum(absolute value of beta). Some embodiments include: cumulative biomarker score=number of analytes that deviate 1 sigma from mean/total analytes. Some embodiments include a calculation such as: cumulative biomarker score=Sum(absolute value of beta). Some embodiments include: cumulative biomarker score=number of analytes that deviate 1 sigma from mean/total analytes. In some embodiments, the cumulative biomarker score is used to determine whether the patient is likely to respond to an HSD17B13 inhibitor. In some embodiments, the cumulative biomarker score is generated using the biomarker measurements. Some embodiments include applying a mathematical algorithm to the obtained biomarker levels to obtain the cumulative biomarker score. In some embodiments, the trained algorithm is or has been generated using regression modeling or machine learning on a training data set.

Prior to treatment, a subject may have an elevated oxylipin measurement, relative to a control subject. The oxylipin measurement may be reduced upon treatment. The oxylipin measurement may include an esterified oxylipin. The oxylipin measurement may include a non-esterified oxylipin.

### Identifying Liver Disease Risk

Provided herein, in some embodiments, are methods for identifying a risk that a patient has or will develop a liver disease. Some embodiments include obtaining one or more biomarker measurements such as an oxylipin measurement. Some embodiments include receiving one or more biomarker measurements. In some embodiments, the biomarker measurements are obtained in a sample from a patient. Some embodiments include obtaining one or more biomarker measurements in at least one sample from a patient. Some embodiments include receiving one or more biomarker measurements in at least one sample from a patient. The liver disease may be a liver disease described herein. Some embodiments include evaluating a disease or likelihood of disease development in the subject state based on the oxylipin measurement.

The disease may be a liver disease. Some examples of liver diseases may include liver inflammation, liver fibrosis, cholestasis, a gall bladder disease, a biliary tree disease, alcoholic liver disease, or non-alcoholic steatohepatitis.

Disclosed herein, in some embodiments, are methods for identifying a risk that a patient has or will develop a liver disease. Some embodiments include obtaining or receiving one or more biomarker measurements in at least one sample from a patient, the biomarkers comprising oxylipins such as those in Table 1.

Disclosed herein, in some embodiments, are methods for identifying a risk that a patient has or will develop a liver disease. Some embodiments include identifying, based on the obtained or received measurements, a risk that the patient has or will develop a liver disease. Some embodiments include identifying the patient as having the liver disease. Some embodiments include identifying the patient as likely having the liver disease. Some embodiments include identifying the patient as at risk of developing the liver disease. Some embodiments include identifying the patient as not having the liver disease. Some embodiments include identifying the patient as unlikely to have the liver disease. Some embodiments include identifying the patient as not at risk of developing the liver disease.

Provided herein, in some embodiments, are methods for identifying a risk that a patient has or will develop a liver disease, comprising: obtaining or receiving measurements of one or more biomarkers in at least one sample from a patient, the biomarkers comprising oxylipins such as those in Table 1; and identifying, based on the obtained or received measurements, a risk that the patient has or will develop a liver disease.

Disclosed herein, in some embodiments, are methods for identifying a risk that a patient has or will develop a liver disease. Some embodiments include comparing the one or more biomarker measurements such as oxylipin measurements to a control measurement or threshold for each biomarker. Some embodiments include comparing the one or more biomarker measurements to a control measurement. Some embodiments include comparing the one or more biomarker measurements to a threshold for each biomarker. In some embodiments, the control measurement is obtained from a patient population such as a general patient population.

Disclosed herein, in some embodiments, are methods for identifying a patient likely to benefit from treatment with an HSD17B13 inhibitor based on obtained or received biomarker measurements such as oxylipin measurements. Some embodiments include, for each biomarker measurement, determining a variance of the biomarker measurement from the control measurement, or determining whether the biomarker measurement exceeds the threshold. Some embodiments include, for each biomarker measurement, determining a variance of the biomarker measurement from the control measurement. Some embodiments include, for each biomarker measurement, determining whether the biomarker measurement exceeds the threshold.

Disclosed herein, in some embodiments, are methods for identifying a patient likely to benefit from treatment with an HSD17B13 inhibitor based on obtained or received biomarker measurements such as oxylipin measurements. Some embodiments include, for each biomarker measurement, determining whether the biomarker measurement is predictive based on the variance of the biomarker measurement from the control measurement, or based on whether the biomarker measurement exceeds the threshold. Some embodiments include, for each biomarker measurement, determining whether the biomarker measurement is predictive based on the variance of the biomarker measurement from the control measurement. Some embodiments include, for each biomarker measurement, determining whether the biomarker measurement is predictive based on whether the biomarker measurement exceeds the threshold.

Disclosed herein, in some embodiments, are methods for identifying a patient likely to benefit from treatment with an HSD17B13 inhibitor based on obtained or received biomarker measurements such as oxylipin measurements. In some embodiments, a biomarker measurement is predictive. In some embodiments, a biomarker measurement is predictive when the variance is at least 1 σ from a mean of the patient population (e.g. general patient population).

Disclosed herein, in some embodiments, are methods for identifying a patient likely to benefit from treatment with an HSD17B13 inhibitor based on obtained or received biomarker measurements. In some embodiments, one or more oxylipins in Table 1 are predictive when they are greater in the patient than the control measurement or threshold.

Some embodiments include determining a number of biomarker measurements such as oxylipin measurements that are predictive. Some embodiments include identifying the patient as at risk of having or developing the liver disease when the number of biomarker measurements that are predictive is at least 1, at least 2, at least 3, at least 4, at least 5, at least 6, at least 7, at least 8, at least 9, or at least 10, or as not at risk of having or developing the liver disease when the number of biomarker measurements that are predictive is less than 1, less than 2, less than 3, less than 4, less than 5, less than 6, less than 7, less than 8, less than 9, or less than 10.

Some embodiments include identifying a cumulative biomarker panel deviation of the patient from a cumulative mean of the patient population. Some embodiments include identifying the patient as at risk of having or developing the liver disease when the cumulative biomarker panel of the patient deviates sufficiently from the cumulative mean of the patient population, and identifying the patient as not at risk of having or developing the liver disease when the cumulative biomarker panel of the patient does not deviate sufficiently from the cumulative mean of the patient population. In some embodiments, the deviation is sufficient if it is at least 1 σ from a mean of the patient population. Some embodiments include providing the treatment with the HSD17B13 inhibitor to the patient based on the identified risk that the patient has or will develop a liver disease.

Some embodiments relate to a cumulative biomarker panel deviation, or a cumulative biomarker score. The cumulative biomarker panel deviation may be determined through summation of the deviation from the mean. The cumulative biomarker panel deviation may include rank of a number of analytes as deviating from the mean. The cumulative biomarker score may be determined through summation of the deviation from the mean. The cumulative biomarker score may include rank of a number of analytes as deviating from the mean. Some embodiments include a calculation such as: cumulative biomarker score=Sum(absolute value of beta). Some embodiments include: cumulative biomarker score=number of analytes that deviate 1 sigma from mean/total analytes. Some embodiments include a calculation such as: cumulative biomarker score=Sum(absolute value of beta). Some embodiments include: cumulative biomarker score=number of analytes that deviate 1 sigma from mean/total analytes.

In some embodiments, the cumulative biomarker score is used to determine whether the patient is at risk of having or developing a liver disease. In some embodiments, the cumulative biomarker score is generated using the biomarker measurements. Some embodiments include applying a mathematical algorithm to the obtained biomarker levels to obtain the cumulative biomarker score. In some embodiments, the trained algorithm is or has been generated using regression modeling or machine learning on a training data set.

### Methods of Evaluating an HSD17B13 Inhibitor Treatment

Provided herein, in some embodiments, are methods for evaluating an HSD17B13 inhibitor treatment of a patient. Some embodiments include obtaining one or more biomarker measurements such as an oxylipin measurement. Some embodiments include receiving one or more biomarker measurements. In some embodiments, the biomarker measurements are obtained in a sample from a patient. Some embodiments include obtaining one or more biomarker measurements in at least one sample from a patient. Some embodiments include receiving one or more biomarker measurements in at least one sample from a patient. Some embodiments include evaluating a HSD17B13 inhibitor treatment of the subject based on the oxylipin measurement. Oxylipin measurements may be useful for identifying target engagement (e.g. HSD17B13 engagement) by an HSD17B13 inhibitor, or for determining a degree, or an amount of target engagement by an HSD17B13 inhibitor.

Provided herein, in some embodiments, are methods comprising obtaining or receiving biomarker measurements such as oxylipin measurements in at least one sample from a patient. In some embodiments, the sample is a from a patient receiving an HSD17B13 inhibitor treatment. In some embodiments, the patient received the HSD17B13 inhibitor treatment before the sample is obtained from the patient. In some embodiments, the patient received the HSD17B13 inhibitor treatment 1 day, 2 days, 3 days, 4 days, 5 days, 6 days, 1 week, 2 weeks, 3 weeks, 4 weeks, 1 month, 2 months, 3 months, 4 months, 5 months, 6 months, 7 months, 8 months, 9 months, 10 months, 11 months, 1 year, 2 years, 3 years, 4 years, or 5 years, or a range of times defined by any two of the aforementioned time periods, before the sample was obtained from the patient. In some embodiments, the patient received the HSD17B13 inhibitor treatment at least 1 day, at least 2 days, at least 3 days, at least 4 days, at least 5 days, at least 6 days, at least 1 week, at least 2 weeks, at least 3 weeks, at least 4 weeks, at least 1 month, at least 2 months, at least 3 months, at least 4 months, at least 5 months, at least 6 months, at least 7 months, at least 8 months, at least 9 months, at least 10 months, at least 11 months, at least 1 year, at least 2 years, at least 3 years, at least 4 years, or at least 5 years, before the sample was obtained from the patient. In some embodiments, the patient received the HSD17B13 inhibitor treatment no more than 1 day, no more than 2 days, no more than 3 days, no more than 4 days, no more than 5 days, no more than 6 days, no more than 1 week, no more than 2 weeks, no more than 3 weeks, no more than 4 weeks, no more than 1 month, no more than 2 months, no more than 3 months, no more than 4 months, no more than 5 months, no more than 6 months, no more than 7 months, no more than 8 months, no more than 9 months, no more than 10 months, no more than 11 months, no more than 1 year, no more than 2 years, no more than 3 years, no more than 4 years, or no more than 5 years, before the sample was obtained from the patient.

Provided herein, in some embodiments, are methods comprising obtaining or receiving biomarker measurements in at least one sample from a patient receiving an HSD17B13 inhibitor treatment. In some embodiments, the patient received the HSD17B13 inhibitor treatment 1, 2, 3, 4, 5, 6, 7, 8, 9, 10, 15, 20, 25, 30, 35, 40, 45, 50, or more times, or a range of times defined by any of two the aforementioned integers, before the sample was obtained from the patient. The patient may have been receiving ongoing HSD17B13 inhibitor treatment, or the HSD17B13 inhibitor treatment may have ended before the sample was obtained from the patient.

Disclosed herein, in some embodiments, are methods for evaluating an HSD17B13 inhibitor treatment of a patient. Some embodiments include obtaining or receiving one or more biomarker measurements in at least one sample from a patient from a patient receiving an HSD17B13 inhibitor treatment, the biomarkers comprising oxylipins such as those in Table 1.

Disclosed herein, in some embodiments, are methods for evaluating an HSD17B13 inhibitor treatment of a patient. Some embodiments include identifying, based on the obtained or received measurements, the patient as likely to benefit from a treatment with an HSD17B13 inhibitor or as not likely to benefit from the treatment. The HSD17B13 inhibitor may be any HSD17B13 inhibitor described herein.

Provided herein, in some embodiments, are methods for evaluating an HSD17B13 inhibitor treatment of a patient, comprising: receiving or obtaining measurements of biomarkers in at least one sample from a patient receiving an HSD17B13 inhibitor treatment, the biomarkers comprising oxylipins such as those in Table 1; and evaluating the HSD17B13 inhibitor treatment of the patient based on the obtained or received measurements.

Disclosed herein, in some embodiments, are methods for evaluating an HSD17B13 inhibitor treatment of a patient. Some embodiments include comparing the one or more biomarker measurements such as oxylipin measurements to a control measurement or threshold for each biomarker. Some embodiments include comparing the one or more biomarker measurements to a control measurement. Some embodiments include comparing the one or more biomarker measurements to a threshold for each biomarker.

Disclosed herein, in some embodiments, are methods for evaluating an HSD17B13 inhibitor treatment of a patient based on obtained or received biomarker measurements such as oxylipin measurements. Some embodiments include, for each biomarker measurement, determining whether the biomarker measurement is significantly different from the baseline measurement. Some embodiments include, for each biomarker measurement, determining a predictive value of the biomarker measurement based on whether the biomarker measurement is significantly different from the baseline measurement. In some embodiments, a biomarker measurement is significantly different from the baseline measurement when the biomarker measurement is at least 5% different from the baseline measurement. In some embodiments, a biomarker measurement is significantly different from the baseline measurement when the biomarker measurement is at least 10% different from the baseline measurement. In some embodiments, a biomarker measurement is significantly different from the baseline measurement when the biomarker measurement is at least 15% different, at least 20% different, or at least 25% different, from the baseline measurement.

Disclosed herein, in some embodiments, are methods for evaluating an HSD17B13 inhibitor treatment of a patient based on obtained or received biomarker measurements. In some embodiments, the biomarkers include oxylipins in Table 1 have a predictive value that indicates that the HSD17B13 inhibitor treatment of the patient is effective when the measurement is significantly less than the baseline measurement. In some embodiments, the oxylipins indicate that the inhibitor treatment is ineffective or needs to be adjusted.

Disclosed herein, in some embodiments, are methods for evaluating an HSD17B13 inhibitor treatment of a patient based on obtained or received biomarker measurements such as oxylipin measurements. Some embodiments include determining a number of biomarker measurements with a predictive value that indicates that the HSD17B13 inhibitor treatment of the patient is effective. In some embodiments, the evaluation is that the HSD17B13 inhibitor treatment of the patient is effective when the number of biomarker measurements with predictive value is at least 1, at least 2, at least 3, at least 4, at least 5, at least 6, at least 7, at least 8, at least 9, or at least 10, or wherein the evaluation is that the HSD17B13 inhibitor treatment of the patient is ineffective when the number of biomarker measurements with predictive value is less than 1, less than 2, less than 3, less than 4, less than 5, less than 6, less than 7, less than 8, less than 9, or less than 10.

Disclosed herein, in some embodiments, are methods for evaluating an HSD17B13 inhibitor treatment of a patient based on obtained or received biomarker measurements such as oxylipin measurements. Some embodiments include identifying a cumulative biomarker value for the patient before and after the HSD17B13 inhibitor treatment. In some embodiments, the evaluation is that the HSD17B13 inhibitor treatment of the patient is effective when the cumulative biomarker value after treatment deviates sufficiently the cumulative biomarker value before treatment, and wherein the evaluation is that the HSD17B13 inhibitor treatment of the patient is ineffective when the cumulative biomarker value after treatment does not deviate sufficiently the cumulative biomarker value before treatment. In some embodiments, the deviation is sufficient if it is at least 10%, 20%, 30%, 40%, 50%, 75%, 100%, 150%, 200%, 250%, 500%, 750%, or 1000%, or greater.

Disclosed herein, in some embodiments, are methods for evaluating an HSD17B13 inhibitor treatment of a patient based on obtained or received biomarker measurements such as oxylipin measurements. Some embodiments include increasing a dose or amount of HSD17B13 inhibitor in a subsequent HSD17B13 inhibitor treatment, or recommending an increase in the dose or amount of a subsequent HSD17B13 inhibitor treatment, based on the evaluation of the HSD17B13 inhibitor treatment. Some embodiments include increasing a dose or amount of HSD17B13 inhibitor in a subsequent HSD17B13 inhibitor treatment, or recommending an increase in the dose or amount of a subsequent HSD17B13 inhibitor treatment, when the HSD17B13 inhibitor treatment of the patient is successful or unsuccessful. Some embodiments include decreasing a dose or amount of HSD17B13 inhibitor in a subsequent HSD17B13 inhibitor treatment, or recommending a decrease in the dose or amount of a subsequent HSD17B13 inhibitor treatment, based on the evaluation of the HSD17B13 inhibitor treatment. Some embodiments include decreasing a dose or amount of HSD17B13 inhibitor in a subsequent HSD17B13 inhibitor treatment, or recommending a decrease in the dose or amount of a subsequent HSD17B13 inhibitor treatment, when the HSD17B13 inhibitor treatment of the patient is successful or unsuccessful. Some embodiments include adjusting the dose until the oxylipin measurements are similar to those found in patients with inactivating HSD17B13 mutations.

### Determining HSD17B13 Activity

Disclosed herein, in some embodiments, are methods such as methods of treating a subject. The methods may relate to a 170-Hydroxysteroid dehydrogenase type 13 (HSD17B13) activity. The methods may be used to identify a dose of an HSD17B13 inhibitor to administer to a subject, determine whether a subject is likely to benefit from treatment with an HSD17B13 inhibitor, or determine a subject's risk of developing a liver disease based on the HSD17B13 activity.

Some embodiments include administering an HSD17B13 substrate to a subject. The substrate may include an oxylipin or oxylipin precursor. A precursor may include a parent lipid in Table 2. Some embodiments include administering to a subject a first amount of an HSD17B13 substrate. Some embodiments include determining an amount of a reaction product of the HSD17B13 substrate. Some embodiments include determining an amount of a reaction product of the HSD17B13 substrate in a sample obtained from the subject. In some embodiments, the sample is obtained from the subject after administering the first amount of the HSD17B13 substrate to the subject. Some embodiments include determining a second amount of the HSD17B13 substrate. Some embodiments include determining a second amount of the HSD17B13 substrate in a sample obtained from the subject. In some embodiments, the sample is obtained from the subject after administering the first amount of the HSD17B13 substrate to the subject. Some embodiments include determining an amount of a reaction product of the HSD17B13 substrate and the second amount of the HSD17B13 substrate in the same sample. Some embodiments include determining an amount of a reaction product of the HSD17B13 substrate and the second amount of the HSD17B13 substrate in separate samples. Disclosed herein, in some embodiments, are methods of treatment, comprising: administering to a subject a first amount of an HSD17B13 substrate; and determining an amount of a reaction product of the HSD17B13 substrate in a sample obtained from the subject, or determining a second amount of the HSD17B13 substrate in a sample obtained from the subject after administering the first amount of the HSD17B13 substrate to the subject.

Some embodiments include administering an HSD17B13 substrate precursor such as an oxylipin precursor to a subject. Some embodiments include administering to a subject a first amount of an HSD17B13 substrate precursor. Some embodiments include determining an amount of the HSD17B13 substrate before administering the HSD17B13 substrate precursor to a subject. Some embodiments include determining an amount of the HSD17B13 substrate after administering the HSD17B13 substrate precursor to a subject. Some embodiments include determining an amount of a reaction product of the HSD17B13 substrate. Any of the determinations (e.g. measurements) may be made in a sample obtained from the subject. A sample for measurement may be obtained from the subject before or after administering the first amount of the HSD17B13 substrate precursor to the subject. Some embodiments include determining a second amount of the HSD17B13 substrate precursor. Some embodiments include determining a second amount of the HSD17B13 substrate precursor in a sample obtained from the subject. In some embodiments, the sample is obtained from the subject after administering the first amount of the HSD17B13 substrate precursor to the subject. Some embodiments include administering to a subject a first amount of an HSD17B13 substrate precursor; determining an amount of the HSD17B13 substrate formed; and determining an amount of a reaction product of the HSD17B13 substrate in a sample obtained from the subject. Some embodiments include determining a second amount of the HSD17B13 substrate precursor in a sample obtained from the subject after administering the first amount of the HSD17B13 substrate precursor to the subject.

In some embodiments, the subject has already been administered the HSD17B13 substrate (or substrate precursor). Some such embodiments include determining an amount of a reaction product of the HSD17B13 substrate in a sample obtained from a subject that has been administered an HSD17B13 substrate. Some such embodiments include determining a second amount of the HSD17B13 substrate in a sample obtained from a subject that has been administered a first amount of an HSD17B13 substrate.

The substrate may include an HSD17B13 substrate such as an oxylipin or oxylipin precursor. Examples of oxylipins are included in Table 1. The reaction product may include an HSD17B13 substrate described herein. In some embodiments, the reaction product comprises an oxidized form of the HSD17B13 substrate. In some embodiments, the HSD17B13 substrate comprises a primary alcohol, and the reaction product comprises an aldehyde. In some embodiments, the HSD17B13 substrate comprises a secondary alcohol, and the reaction product comprises a ketone.

Some embodiments include obtaining a sample or a first sample from a subject. In some embodiments, the sample is obtained from the subject 30 min, 1 hr, 2 hr, 5 hr, 6 hr, 8 hr, 10 hr, 12 hr, 15 hr, 18 hr, 24 hr, 2 days, 3 days, 4 days, 5 days, 6 days, or 7 days, or a range of times defined by any two of the aforementioned times, after administering the first amount of the HSD17B13 substrate such as an oxylipin or oxylipin precursor to the subject. In some embodiments, the sample is obtained from the subject at least 30 min, at least 1 hr, at least 2 hr, at least 5 hr, at least 6 hr, at least 8 hr, at least 10 hr, at least 12 hr, at least 15 hr, at least 18 hr, at least 24 hr, at least 2 days, at least 3 days, at least 4 days, at least 5 days, at least 6 days, or at least 7 days after administering the first amount of the HSD17B13 substrate to the subject. In some embodiments, the sample is obtained from the subject no more than 30 min, no more than 1 hr, no more than 2 hr, no more than 5 hr, no more than 6 hr, no more than 8 hr, no more than 10 hr, no more than 12 hr, no more than 15 hr, no more than 18 hr, no more than 24 hr, no more than 2 days, no more than 3 days, no more than 4 days, no more than 5 days, no more than 6 days, or no more than 7 days, after administering the first amount of the HSD17B13 substrate to the subject.

Some embodiments include administration or coadministration of an HSD17B13 inhibitor to the subject with or at a similar time as the HSD17B13 substrate such as an oxylipin or oxylipin precursor. Some embodiments include administering or co-administering a first amount of an HSD17B13 inhibitor to the subject with the administration of the first amount of the HSD17B13 substrate. The administration or coadministration of an HSD17B13 inhibitor may be used to determine an amount by which the HSD17B13 inhibitor inhibits HSD17B13 activity in the subject. This may be useful for obtaining the correct dosing of the subject of the HSD17B13 inhibitor.

Some embodiments include determining an HSD17B13 enzyme activity. The HSD17B13 enzyme activity may be determined based on the amount of the reaction product measured in the sample obtained after administration of the HSD17B13 substrate to the subject. The HSD17B13 enzyme activity may be determined based on the amount of HSD17B13 substrate such as an oxylipin or oxylipin precursor measured in the sample obtained after administration of the HSD17B13 substrate to the subject. Some embodiments include determining an HSD17B13 enzyme activity based on the amount of the reaction product or second amount of the HSD17B13 substrate in the sample. Such measurements may be made over time, and an area under the curve may be obtained.

Some embodiments include administering an HSD17B13 substrate (such as a first amount of the HSD17B13 substrate) to a subject. In some embodiments, the administration of the HSD17B13 substrate to the subject is intravenous. For example, the HSD17B13 substrate may be infused into the bloodstream of the subject. The HSD17B13 substrate may be injected into the subject or into the subject's bloodstream. In some embodiments, the administration of the HSD17B13 substrate to the subject is oral. In some embodiments, the administration of the first amount of the HSD17B13 substrate is intravenous or oral.

Some embodiments include administering an amount of an HSD17B13 substrate (such as a first amount of the HSD17B13 substrate) to a subject. Various amounts of the HSD17B13 substrate may be administered. For example, micro amounts may be administered to the subject. In some embodiments, 5 μg, 10 μg, 25 μg, 50 μg, 75 μg, 100 μg, 150 μg, 200 μg, 250 μg, 500 μg, 750 μg, or 1000 μg, or a range defined by any of the aforementioned amounts, of the HSD17B13 substrate is administered to the subject. In some embodiments, about 5 μg, about 10 μg, about 25 μg, about 50 μg, about 75 μg, about 100 μg, about 150 μg, about 200 μg, about 250 μg, about 500 μg, about 750 μg, or about 1000 μg, of the HSD17B13 substrate is administered to the subject. In some embodiments, at least 5 μg, at least 10 μg, at least 25 μg, at least 50 μg, at least 75 μg, at least 100 μg, at least 150 μg, at least 200 μg, at least 250 μg, at least 500 μg, at least 750 μg, or at least 1000 μg, of the HSD17B13 substrate is administered to the subject. In some embodiments, no more than 5 μg, no more than 10 μg, no more than 25 μg, no more than 50 μg, no more than 75 μg, no more than 100 μg, no more than 150 μg, no more than 200 μg, no more than 250 μg, no more than 500 μg, no more than 750 μg, or no more than 1000 μg, of the HSD17B13 substrate is administered to the subject. In some embodiments, the first amount of the HSD17B13 substrate is 10-1000 μg of the HSD17B13 substrate. In some embodiments, the first amount of the HSD17B13 substrate is about 100 μg of the HSD17B13 substrate.

Some examples of oxylipin precursors may include linoleic acid or alpha-linolenic acid. Some embodiments further include administering linoleic acid to the subject prior to obtaining the oxylipin measurement, or wherein the subject has been administered linoleic acid prior to receiving the oxylipin measurement. In some embodiments, the administered linoleic acid comprises a label. Some embodiments further include administering alpha-linoleic acid to the subject prior to obtaining the oxylipin measurement, or wherein the subject has been administered alpha-linoleic acid prior to receiving the oxylipin measurement. In some embodiments, the administered alpha-linoleic acid comprises a label. In some embodiments, the label comprises an isotopic label. In some embodiments, the isotopic label comprises ¹³C or ²H. In some embodiments, the oxylipin comprises the label.

In some embodiments, the HSD17B13 substrate and the HSD17B13 reaction product are labeled. In some embodiments, the HSD17B13 substrate and the HSD17B13 reaction product are radiolabeled. In some embodiments, the radiolabeled HSD17B13 substrate and reaction product comprise ¹³ or ²H. In some embodiments, the HSD17B13 substrate is labeled. In some embodiments, the HSD17B13 substrate is radiolabeled. In some embodiments, the radiolabeled HSD17B13 substrate comprises ¹³C. In some embodiments, the radiolabeled HSD17B13 substrate comprises ²H. In some embodiments, the radiolabeled HSD17B13 substrate comprises ¹³C and ²H. In some embodiments, determining the second amount of the HSD17B13 substrate comprises measuring the radiolabeled HSD17B13 substrate in the sample. In some embodiments, the HSD17B13 reaction product is labeled. In some embodiments, the HSD17B13 reaction product is radiolabeled. In some embodiments, the radiolabeled HSD17B13 reaction product comprises ¹³C. In some embodiments, the radiolabeled HSD17B13 reaction product comprises ²H. In some embodiments, the radiolabeled HSD17B13 reaction product comprises ¹³C and ²H. In some embodiments, determining the amount of the reaction product comprises measuring the radiolabeled reaction product in the sample.

In some embodiments, identifying the HSD17B13 enzyme activity based on the amount of the reaction product or second amount of the HSD17B13 substrate in the sample, comprises detecting increases in oxidized ¹³C or ²H labeled product over time after the administration of the first amount of the HSD17B13 substrate, or comprises detecting decreases in ¹³C or ²H labeled substrate overtime after the administration of the first amount of the HSD17B13 substrate. In some embodiments, identifying the HSD17B13 enzyme activity based on the amount of the reaction product in the sample, comprises detecting increases in oxidized ¹³C or ²H labeled product over time after the administration of the first amount of the HSD17B13 substrate. In some embodiments, identifying the HSD17B13 enzyme activity based on the second amount of the HSD17B13 substrate in the sample, comprises detecting decreases in ¹³C or ²H labeled substrate over time after the administration of the first amount of the HSD17B13 substrate.

Some embodiments include determining the amount of the reaction product of the HSD17B13 substrate in the sample. Some embodiments include determining the second amount of the HSD17B13 substrate in the sample. Some embodiments include determining the amount of the reaction product of the HSD17B13 substrate and the second amount of the HSD17B13 substrate in the sample.

Some embodiments include determining a second amount of the reaction product of the HSD17B13 substrate in a second sample. Some embodiments include determining a third amount of the HSD17B13 substrate in the second sample. In some embodiments, the second sample is obtained from the subject at a second time after administering the first amount of the HSD17B13 substrate to the subject. Some embodiments include determining a second amount of the reaction product of the HSD17B13 substrate in a second sample, or determining a third amount of the HSD17B13 substrate in the second sample, wherein the second sample is obtained from the subject at a second time after administering the first amount of the HSD17B13 substrate to the subject.

In some embodiments, the second time comprises 30 min, 1 hr, 2 hr, 5 hr, 6 hr, 8 hr, 10 hr, 12 hr, 15 hr, 18 hr, 24 hr, 2 days, 3 days, 4 days, 5 days, 6 days, or 7 days, or a range of time periods defined by any two of the aforementioned time periods. In some embodiments, the second time comprises at least 30 min, at least 1 hr, at least 2 hr, at least 5 hr, at least 6 hr, at least 8 hr, at least 10 hr, at least 12 hr, at least 15 hr, at least 18 hr, at least 24 hr, at least 2 days, at least 3 days, at least 4 days, at least 5 days, at least 6 days, or at least 7 days. In some embodiments, the second time comprises no more than 30 min, no more than 1 hr, no more than 2 hr, no more than 5 hr, no more than 6 hr, no more than 8 hr, no more than 10 hr, no more than 12 hr, no more than 15 hr, no more than 18 hr, no more than 24 hr, no more than 2 days, no more than 3 days, no more than 4 days, no more than 5 days, no more than 6 days, or no more than 7 days.

Some embodiments include comparing the second amount of the reaction product to the first amount of the reaction product. Some embodiments include comparing the third amount of the HSD17B13 substrate to the second amount of the HSD17B13 substrate. Some embodiments include comparing the second amount of the reaction product to the first amount of the reaction product, or comparing the third amount of the HSD17B13 substrate to the second amount of the HSD17B13 substrate.

Some embodiments include generating an area under the curve of the first and second amounts of the reaction product. Some embodiments include generating an area under the curve of the second and third amounts of the HSD17B13 substrate. Some embodiments include generating an area under the curve of the first and second amounts of the reaction product, or generating an area under the curve of the second and third amounts of the HSD17B13 substrate.

Some embodiments include comparing a first area under the curve of the first and second amounts of the reaction product. Some embodiments include comparing a first area under the curve of the first and second amounts of the reaction product to a second area under the curve of amounts of the reaction product or HSD17B13 substrate. Some embodiments include comparing the second and third amounts of the HSD17B13 substrate. Some embodiments include comparing the second and third amounts of the HSD17B13 substrate to a second area under the curve of amounts of the reaction product or HSD17B13 substrate. In some embodiments, the first area under the curve is generated when the HSD17B13 substrate is coadministered to the subject with an HSD17B13 inhibitor. In some embodiments, the second area under the curve is generated when the HSD17B13 substrate is not coadministered to the subject with the HSD17B13 inhibitor. Some embodiments include comparing a first area under the curve of the first and second amounts of the reaction product, or the second and third amounts of the HSD17B13 substrate, to a second area under the curve of amounts of the reaction product or HSD17B13 substrate, wherein the first area under the curve is generated when the HSD17B13 substrate is coadministered to the subject with an HSD17B13 inhibitor, and wherein the second area under the curve is generated when the HSD17B13 substrate is not coadministered to the subject with the HSD17B13 inhibitor. In some embodiments, the first area under the curve is generated when the HSD17B13 substrate is coadministered to the subject with a first amount of an HSD17B13 inhibitor. In some embodiments, the second area under the curve is generated when the HSD17B13 substrate is coadministered to the subject with a second amount of the HSD17B13 inhibitor. Some embodiments include comparing a first area under the curve of the first and second amounts of the reaction product, or the second and third amounts of the HSD17B13 substrate, to a second area under the curve of amounts of the reaction product or HSD17B13 substrate, wherein the first area under the curve is generated when the HSD17B13 substrate is coadministered to the subject with a first amount of an HSD17B13 inhibitor, and wherein the second area under the curve is generated when the HSD17B13 substrate is coadministered to the subject with a second amount of the HSD17B13 inhibitor.

Some embodiments include comparing the HSD17B13 enzyme activity to a baseline HSD17B13 enzyme activity obtained without co-administering the HSD17B13 inhibitor to the subject with the HSD17B13 substrate. Some embodiments include administering a second amount of the HSD17B13 inhibitor to the subject based on the HSD17B13 enzyme activity. Some embodiments include comparing the HSD17B13 enzyme activity to the baseline HSD17B13 enzyme activity. Some embodiments include determining based on the comparison whether to increase, decrease, or not change the second amount of the HSD17B13 inhibitor relative to the first amount of the HSD17B13 inhibitor. Some embodiments include comparing the HSD17B13 enzyme activity to the baseline HSD17B13 enzyme activity, and determining based on the comparison whether to increase, decrease, or not change the second amount of the HSD17B13 inhibitor relative to the first amount of the HSD17B13 inhibitor. In some embodiments, the second amount of the HSD17B13 inhibitor is greater than the first amount of the HSD17B13 inhibitor. In some embodiments, the second amount of the HSD17B13 inhibitor is less than the first amount of the HSD17B13 inhibitor. In some embodiments, the second amount of the HSD17B13 inhibitor is about the same as the first amount of the HSD17B13 inhibitor.

Some embodiments include providing the subject with a ¹³C or ²H labeled HSD17B13 substrate, such as an oxylipin or oxylipin precursor, with different doses of the HSD17B13 inhibitor to obtain dose levels that inhibit labeled product formation at 25%, 50%, 75%, 85%, 90%, or 95%. Some embodiments include administering a labeled HSD17B13 substrate to a subject. The subject may also be coadministered one or more doses of an HSD17B13 inhibitor. Some embodiments include determining what amounts of inhibition each of the one or more doses (e.g. multiple doses) has upon HSD17B13 activity within the subject. The HSD17B13 activity may be determined at each dose of the HSD17B13 inhibitor. Thus, the optimal dose may be provided based on what amount each dose inhibits HSD17B13 activity, and what amount of HSD17B13 activity inhibition is desired.

Some embodiments include performing assays to aid in selection of clinical doses that sufficiently inhibit the subject's HSD17B13. For example, some embodiments include administering, with the first amount of the HSD17B13 substrate, a first amount of an HSD17B13 inhibitor, wherein the first amount of the HSD17B13 inhibitor inhibits reaction product formation by about 25%, about 50%, about 75%, about 85%, about 90%, or about 95%. Some embodiments include administering, with the first amount of the HSD17B13 substrate, a first amount of an HSD17B13 inhibitor. In some embodiments, the first amount of the HSD17B13 inhibitor inhibits reaction product formation by an amount. Some embodiments include administering, with the first amount of the HSD17B13 substrate, a first amount of an HSD17B13 inhibitor, wherein the first amount of the HSD17B13 inhibitor inhibits reaction product formation by an amount. In some embodiments, the first amount of the HSD17B13 inhibitor inhibits reaction product formation by 25%, 50%, 75%, 85%, 90%, or 95%, or a range of percentages defined by any two of the aforementioned percentages. In some embodiments, the first amount of the HSD17B13 inhibitor inhibits reaction product formation by about 25%, about 50%, about 75%, about 85%, about 90%, or about 95%. In some embodiments, the first amount of the HSD17B13 inhibitor inhibits reaction product formation by at least 25%, at least 50%, at least 75%, at least 85%, at least 90%, or at least 95%. In some embodiments, the first amount of the HSD17B13 inhibitor inhibits reaction product formation by no greater than 25%, no greater than 50%, no greater than 75%, no greater than 85%, no greater than 90%, or no greater than 95%.

Some embodiments include determining the amount of the HSD17B13 inhibitor that inhibits reaction product formation by a desired amount (such as about 25%, about 50%, about 75%, about 85%, about 90%, or about 95%). In some cases, the desired amount is at least 25%, at least 50%, at least 75%, at least 85%, at least 90%, or at least 95%. In some cases, the desired amount is no greater than 25%, no greater than 50%, no greater than 75%, no greater than 85%, no greater than 90%, or no greater than 95%. Some embodiments include determining the amount of the HSD17B13 inhibitor that inhibits reaction product formation by about 25%, about 50%, about 75%, about 85%, about 90%, or about 95%. Some embodiments include, based on the determined amount of HSD17B13 inhibitor that inhibits reaction product formation by the desired amount, identifying a dose of the HSD17B13 inhibitor to administer to the subject for treatment. Some embodiments include, based on the determined amount of HSD17B13 inhibitor that inhibits reaction product formation by about 25%, about 50%, about 75%, about 85%, about 90%, or about 95%, identifying a dose of the HSD17B13 inhibitor to administer to the subject for treatment. The desired amount may be similar to oxylipin measurements on a readout from a subject with an inactivating mutation in HSD17B13. These methods may enable a clinician to recommend or give the correct dose of an HSD17B13 inhibitor to the subject. In some embodiments, the dose of the HSD17B13 inhibitor is sufficient to treat a liver disease in the patient.

Some embodiments include normalizing the HSD17B13 enzyme activity to obtain a specific HSD17B13 enzyme activity of the subject. In some embodiments, the HSD17B13 enzyme activity is normalized to an amount of the sample to obtain the specific HSD17B13 enzyme activity of the subject. Some embodiments include comparing the HSD17B13 enzyme activity of the subject to a control HSD17B13 enzyme activity measurement, a baseline HSD17B13 enzyme activity measurement, or a threshold HSD17B13 enzyme activity. Some embodiments include comparing the HSD17B13 enzyme activity of the subject to a control HSD17B13 enzyme activity measurement. Some embodiments include comparing the HSD17B13 enzyme activity of the subject to a baseline HSD17B13 enzyme activity measurement. Some embodiments include comparing the HSD17B13 enzyme activity of the subject to a threshold HSD17B13 enzyme activity. In some embodiments, the control HSD17B13 enzyme activity measurement is obtained from a general subject population. In some embodiments, the HSD17B13 enzyme activity of the subject is greater than the control HSD17B13 enzyme activity measurement, baseline HSD17B13 enzyme activity measurement, or threshold HSD17B13 enzyme activity.

Some embodiments include identifying, based on the HSD17B13 enzyme activity of the subject, the subject as likely to benefit from a treatment with an HSD17B13 inhibitor or as not likely to benefit from the treatment. Some embodiments include identifying the subject as likely to benefit from the treatment with the HSD17B13 inhibitor when the HSD17B13 enzyme activity of the subject is greater than the control HSD17B13 enzyme activity measurement, baseline HSD17B13 enzyme activity measurement, or threshold HSD17B13 enzyme activity. In some embodiments, the HSD17B13 enzyme activity of the subject is about the same or lower than the control HSD17B13 enzyme activity measurement, baseline HSD17B13 enzyme activity measurement, or threshold HSD17B13 enzyme activity. Some embodiments include identifying the subject as unlikely to benefit from the treatment with the HSD17B13 inhibitor when the HSD17B13 enzyme activity of the subject is about the same or lower than the control HSD17B13 enzyme activity measurement, baseline HSD17B13 enzyme activity measurement, or threshold HSD17B13 enzyme activity.

Some embodiments include identifying, based on the HSD17B13 enzyme activity measurement, a risk that the subject has or will develop a liver disease. Some embodiments include identifying the subject as likely to develop the liver disease when the HSD17B13 enzyme activity of the subject is greater than the control HSD17B13 enzyme activity measurement, baseline HSD17B13 enzyme activity measurement, or threshold HSD17B13 enzyme activity. Examples of liver diseases include liver inflammation, liver fibrosis, cholestasis, a gall bladder disease, a biliary tree disease, alcoholic liver disease, or non-alcoholic steatohepatitis.

In some embodiments, measuring the amount of the reaction product or second amount of the HSD17B13 substrate comprises performing liquid chromatography. In some embodiments, measuring the amount of the reaction product or second amount of the HSD17B13 substrate comprises performing high-performance liquid chromatography. In some embodiments, measuring the amount of the reaction product or second amount of the HSD17B13 substrate comprises mass spectrometry. In some embodiments, measuring the amount of the reaction product or second amount of the HSD17B13 substrate comprises performing liquid chromatography, high-performance liquid chromatography or mass spectrometry. Some embodiments include liquid chromatography coupled to mass spectrometry (e.g. LC/MS-MS). Any HSD17B13 substrates or reaction products described herein may be measured using one or more of these methods, among others.

### Patients, Samples and Measurements

Described herein, in some embodiments, are samples such as patient samples. In some embodiments, the sample may be obtained from a subject or patient. The samples may be used or included in the kits or methods described herein. Some embodiments include at least one sample. Some embodiments include a sample. The at least one sample may include a sample.

In some embodiments, the sample is from a subject or patient. In some embodiments, the subject is a vertebrate. In some embodiments, the subject is a mammal. In some embodiments, the subject is a primate. In some embodiments, the patient is a human.

In some embodiments, the patient has a high risk allele for HSD17B13. In some embodiments, the patient has a liver disease. For example, the patient may have ALD or NASH, and a high risk allele for HSD17B13. In some embodiments, the patient has a low risk allele for HSD17B13. The risk allele may be related to a HSD17B13 in the subject. In some embodiments, the patient is homozygous for wild-type HSD17B13. The homozygous wild-type genotype may include a T/T rs72613567 genotype. In some embodiments, the patient is homozygous for an inactive HSD17B13 genotype. The inactive homozygous genotype may include a TA/TA rs72613567 genotype. In some embodiments, the patient is heterozygous for an inactivating/wild-type HSD17B13 genotype. The heterozygous genotype may include a T/TA rs72613567 genotype.

Some embodiments include obtaining biomarker measurements in at least one sample. In some embodiments, the biomarker measurements are obtained from one sample. In some embodiments, the biomarker measurements are obtained from more than one sample. For example, one or more of the biomarker measurements may be obtained in one sample, and another of the biomarkers may be obtained in another sample. Some embodiments include measuring biomarker measurements in one or more samples. In some embodiments, the biomarker measurements are measured in one sample. In some embodiments, the biomarker measurements are measured in more than one sample.

In some embodiments, the tissue comprises liver sample. In some embodiments, the sample comprises a biofluid sample. In some embodiments, the biofluid comprises blood, serum, or plasma. In some embodiments, the sample is a blood sample, a serum sample, or a plasma sample. In some embodiments, the sample comprises a blood sample. In some embodiments, the sample comprises a plasma sample. In some embodiments, the sample comprises a serum sample. In some embodiments, the sample is a blood sample. In some embodiments, the sample is a plasma sample. In some embodiments, the sample is a serum sample. In some embodiments, the sample comprises lipoproteins. In some embodiments, the sample is or includes a sample of lipoproteins. In some embodiments, the lipoproteins comprise low-density lipoproteins (LDLs), very low-density lipoproteins (VLDLs), or high-density lipoproteins (HDLs). In some embodiments, the lipoproteins are enriched in the sample.

The sample may be hydrolyzed. For example, a total plasma or VLDL sample may be hydrolyzed. A blood sample may be hydrolyzed. A plasma sample may be hydrolyzed. A serum sample may be hydrolyzed. A cholesterol sample may be hydrolyzed. A lipoprotein sample may be hydrolyzed. An LDL sample may be hydrolyzed. A VLDL sample may be hydrolyzed. An HDL sample may be hydrolyzed.

Some embodiments include hydrolyzing the sample. This may be done prior to measuring an oxylipin. Some embodiments include hydrolyzing the sample prior to obtaining oxylipin measurements. Hydrolyzing the sample may be useful for releasing an oxylipin from an esterified lipid of the sample. The hydrolysis may be done enzymatically. The hydrolysis may be performed chemically.

In some embodiments, the oxylipin is included in an esterified lipid. In some embodiments, the sample includes lipids, or is a lipid sample. The lipids may be esterified. In some embodiments, the sample includes esterified lipids, or is an esterified lipid sample. The esterified lipids may include an oxylipin. Obtaining an oxylipin may include obtaining esterified lipids. Obtaining an oxylipin measurement may include hydrolyzing esterified lipid to release the oxylipin. In some embodiments, an oxylipin is obtained from an esterified lipid comprising the oxylipin by hydrolyzing the esterified lipid. This may be done prior to measuring the oxylipin. For example, a total plasma sample or VLDL sample may be hydrolyzed to release any oxylipins in the sample, and then the sample may be subjected to LC/MS analysis to obtain oxylipin measurements.

In some embodiments, the sample comprises a tissue. In some embodiments, the sample is a tissue sample. In some embodiments, the tissue sample comprises a liver sample, a gall bladder sample, or a bile duct sample. In some embodiments, the tissue sample comprises a liver sample. In some embodiments, the tissue sample comprises a gall bladder sample. In some embodiments, the tissue sample comprises a bile duct sample.

In some embodiments, the sample is lysed. In some embodiments, the sample is homogenized. In some embodiments, the sample is centrifuged. In some embodiments, the sample is processed to obtain a supernatant or fraction from which to obtain a measurement such as an oxylipin measurement.

Some embodiments include measuring biomarkers such as an oxylipin in a sample. In some embodiments, biomarker measurements are obtained using liquid chromatography, high-performance liquid chromatography or mass spectrometry. In some embodiments, the measurements are obtained using liquid chromatography. In some embodiments, the measurements are obtained using high-performance liquid chromatography. In some embodiments, the measurements are obtained using mass spectrometry. In some embodiments, the biomarkers are measured in the sample using mass spectrometry. Some embodiments include liquid chromatography coupled to mass spectrometry (e.g. LC/MS-MS). In some embodiments, the sample is diluted.

Oxylipins may be measured by a variety of methods. An oxylipin measurement may be generated from a sample after the sample has been treated to isolate or enrich the oxylipin or oxylipins in the sample. Generating an oxylipin measurement may include concentrating, filtering, or centrifuging a sample. Generating an oxylipin measurement may include lipid fractionation. In some embodiments, lipids may be readily separated from other biomolecule types for lipid-specific analysis. As many lipids are strongly hydrophobic, organic solvent extractions and gradient chromatography methods can cleanly separate lipids from other biomolecule-types present within a sample. Lipid data comprising oxylipin measurements may be generated using mass spectrometry. Lipid analysis may then distinguish an oxylipin.

Generating an oxylipin measurement may include using a detection reagent that binds to an oxylipin and yields a detectable signal. After use of a detection reagent that binds to an oxylipin and yields a detectable signal, a readout may be obtained that is indicative of the presence, absence or amount of the oxylipin. Some embodiments include measuring the readout. Generating an oxylipin measurement may include concentrating, filtering, or centrifuging a sample.

Oxylipin measurements may be generated using mass spectrometry, chromatography, liquid chromatography, high-performance liquid chromatography, solid-phase chromatography, a lateral flow assay, an immunoassay, or a combination thereof. An example of a method for generating oxylipin measurements includes using mass spectrometry. Mass spectrometry may include a separation method step such as liquid chromatography (e.g., HPLC). Mass spectrometry may include an ionization method such as electron ionization, atmospheric-pressure chemical ionization, electrospray ionization, or secondary electrospray ionization. Mass spectrometry may include surface-based mass spectrometry or secondary ion mass spectrometry. Another example of a method for generating oxylipin measurements includes nuclear magnetic resonance (NMR). Other examples of methods for generating oxylipin measurements include Fourier-transform ion cyclotron resonance, ion-mobility spectrometry, electrochemical detection (e.g. coupled to HPLC), or Raman spectroscopy and radiolabel (e.g. when combined with thin-layer chromatography).

### HSD17B13 Inhibitors

Described herein, in some embodiments, are inhibitors of HSD17B13 (also referred to herein as “HSD17B13 inhibitors”). Some embodiments relate to compositions including an HSD17B13 inhibitor. The HSD17B13 inhibitors may be used in a method described herein. For example, an HSD17B13 inhibitor may be administered or evaluated in accordance with a method described herein. The HSD17B13 inhibitors may be included in a kit.

Described herein, in some embodiments, are HSD17B13 inhibitors. In some embodiments, the HSD17B13 inhibitor reduces HSD17B13 activity by at least 10%. In some embodiments, the HSD17B13 inhibitor reduces HSD17B13 expression by at least 10%. The reduced activity or expression may be compared to a baseline measurement. The baseline measurement may be measured in a first patient sample, and the reduced activity or expression may be measured in a second patient sample.

Described herein, in some embodiments, are HSD17B13 inhibitors. In some embodiments, the HSD17B13 inhibitor comprises a small molecule, a polypeptide, or an oligonucleotide. In some embodiments, the HSD17B13 inhibitor comprises a small molecule. In some embodiments, the HSD17B13 inhibitor comprises a polypeptide. In some embodiments, the HSD17B13 inhibitor comprises an oligonucleotide.

In some embodiments, the HSD17B13 inhibitor comprises an estradiol mimetic, propyl pyrazole triole, an anti-HSD17B13 antibody or antibody fragment, an oligonucleotide directed against an HSD17B13 encoding oligonucleotide, an antisense oligonucleotide, or an siRNA. In some embodiments, the HSD17B13 inhibitor comprises an estradiol mimetic. In some embodiments, the HSD17B13 inhibitor comprises propyl pyrazole triole. In some embodiments, the HSD17B13 inhibitor comprises an anti-HSD17B13 antibody or antibody fragment. In some embodiments, the HSD17B13 inhibitor comprises an oligonucleotide directed against an HSD17B13 encoding oligonucleotide. For example, the HSD17B13 inhibitor may include a nucleic acid sequence that comprises or is complementary to a nucleic acid sequence encoding HSD17B13. In some embodiments, the HSD17B13 inhibitor comprises an antisense oligonucleotide. In some embodiments, the HSD17B13 inhibitor comprises or an siRNA. In some embodiments, the HSD17B13 inhibitor comprises ARO-HSD. In some embodiments, the HSD17B13 inhibitor comprises ALN-HSD.

Described herein, in some embodiments, are HSD17B13 inhibitors comprising antisense oligonucleotides (ASOs). In some embodiments, the composition comprises an oligonucleotide that inhibits the expression of HSD17B13, wherein the oligonucleotide comprises an antisense oligonucleotide (ASO). In some embodiments, the ASO is 12-30 nucleosides in length. In some embodiments, the ASO is 14-30 nucleosides in length. In some embodiments, the ASO is at least about 10, 11, 12, 13, 14, 15, 16, 17, 18, 19, 20, 21, 22, 23, 24, 25, 26, 27, 28, 29, or 30 nucleosides in length, or a range defined by any of the two aforementioned numbers. In some embodiments, the ASO is 15-25 nucleosides in length. In some embodiments, the ASO is 20 nucleosides in length.

Described herein, in some embodiments, are HSD17B13 inhibitors comprising ASOs. In some embodiments, the composition comprises an oligonucleotide that inhibits the expression of HSD17B13, wherein the oligonucleotide comprises an ASO about 12-30 nucleosides in length and comprising a nucleoside sequence comprising about 12-30 contiguous nucleosides of a full-length human HSD17B13 mRNA sequence; wherein (i) the oligonucleotide comprises a modification comprising a modified nucleoside and/or a modified internucleoside linkage, and/or (ii) the composition comprises a pharmaceutically acceptable carrier.

Described herein, in some embodiments, are HSD17B13 inhibitors comprising small interfering RNAs (siRNAs). In some embodiments, the composition comprises an oligonucleotide that targets HSD17B13, wherein the oligonucleotide comprises an siRNA. In some embodiments, the composition comprises an oligonucleotide that targets HSD17B13, wherein the oligonucleotide comprises an siRNA comprising a sense strand and an antisense strand.

Described herein, in some embodiments, are HSD17B13 inhibitors comprising siRNA. In some embodiments, the composition comprises an oligonucleotide that inhibits the expression of HSD17B13, wherein the oligonucleotide comprises an siRNA comprising a sense strand and an antisense strand, wherein the sense strand is 14-30 nucleosides in length. In some embodiments, the composition comprises a sense strange that is at least about 10, 11, 12, 13, 14, 15, 16, 17, 18, 19, 20, 21, 22, 23, 24, 25, 26, 27, 28, 29, or 30 nucleosides in length, or a range defined by any of the two aforementioned numbers. In some embodiments, the composition comprises an antisense strand is 14-30 nucleosides in length. In some embodiments, the composition comprises an antisense strange that is at least about 10, 11, 12, 13, 14, 15, 15, 17, 18, 19, 20, 21, 22, 23, 24, 25, 26, 27, 28, 29, or 30 nucleosides in length, or a range defined by any of the two aforementioned numbers.

In some embodiments, the HSD17B13 inhibitor comprises:

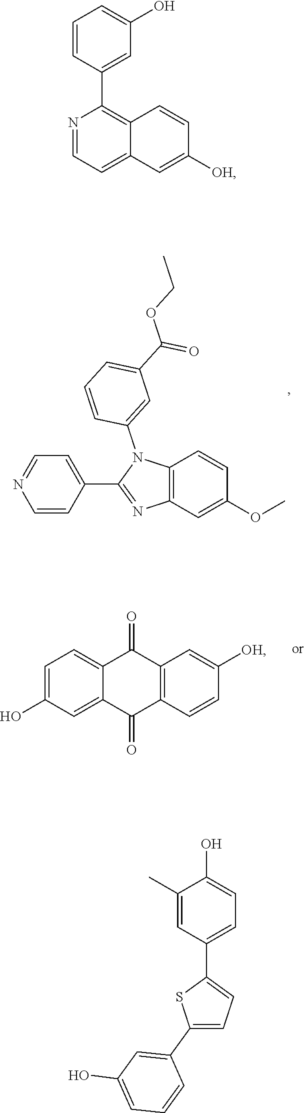 [SMILES: Oc1cccc(-c2nccc3cc(O)ccc23)c1 ; CCOC(=O)c1cccc(-n2c(-c3ccncc3)nc3cc(OC)ccc32)c1 ; O=C1c2ccc(O)cc2C(=O)c2ccc(O)cc21 ; "or" ; Cc1cc(-c2ccc(-c3cccc(O)c3)s2)ccc1O]

or a pharmaceutically acceptable salt or derivative thereof.

In some embodiments, the HSD17B13 inhibitor comprises:

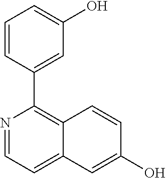 [SMILES: Oc1cccc(-c2nccc3cc(O)ccc23)c1]

In some embodiments, the HSD17B13 inhibitor comprises:

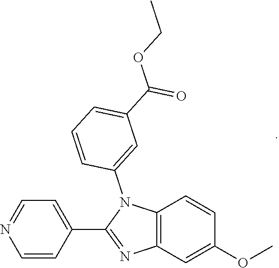 [SMILES: CCOC(=O)c1cccc(-n2c(-c3ccncc3)nc3cc(OC)ccc32)c1]

In some embodiments, the HSD17B13 inhibitor comprises:

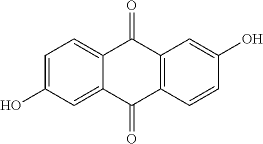 [SMILES: O=C1c2ccc(O)cc2C(=O)c2ccc(O)cc21]

In some embodiments, the HSD17B13 inhibitor comprises:

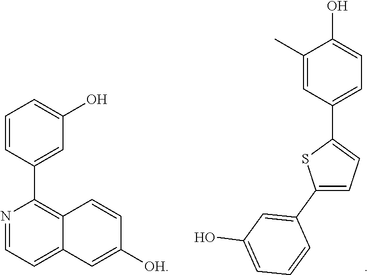 [SMILES: Oc1cccc(-c2nccc3cc(O)ccc23)c1 ; Cc1cc(-c2ccc(-c3cccc(O)c3)s2)ccc1O]

Some examples of HSD17B13 inhibitor are included in CN103520724B, WO2001019798, WO2002096873, WO2019183329, WO2020051207, or US20180291422, each of which is incorporated herein by reference.

Described herein, in some embodiments, are liver disease treatments comprising a composition including an HSD17B13 inhibitor. In some embodiments, the composition is a pharmaceutical composition. In some embodiments, the composition is sterile. In some embodiments, the composition further comprises a pharmaceutically acceptable carrier. In some embodiments, the pharmaceutically acceptable carrier comprises water. In some embodiments, the pharmaceutically acceptable carrier comprises a buffer. In some embodiments, the pharmaceutically acceptable carrier comprises a saline solution. In some embodiments, the pharmaceutically acceptable carrier comprises water, a buffer, or a saline solution. In some embodiments, the composition comprises a liposome. In some embodiments, the pharmaceutically acceptable carrier comprises liposomes, lipids, nanoparticles, proteins, protein-antibody complexes, peptides, cellulose, nanogel, or a combination thereof.

HSD17B13 substrate levels may be increased in response to HSD17B13 inhibition. An esterified oxylipin may be an HSD17B13 substrate. An esterified oxylipin measurement may be increased in response to HSD17B13 inhibition. A non-esterified oxylipin measurement may be increased in response to HSD17B13 inhibition. An esterified oxylipin measurement may be decreased in response to HSD17B13 inhibition. A non-esterified oxylipin may be decreased in response to HSD17B13 inhibition.

### Kits

Provided herein, in some embodiments, are kits. Some embodiments include reagents for measuring a panel of biomarkers such as oxylipins including any of the biomarkers described herein. The kit may include reagents for measuring one or more oxylipins in Table 1. The kit may include an oxylipin precursor. Some examples of oxylipin precursors may include linoleic acid or alpha-linoleic acid. The precursor may be labeled. The label may include an isotopic label such as ¹³C or ²H. Some embodiments include one or more solutions comprising a known concentration of an oxylipin or of an oxylipin precursor. Some embodiments include instructions for using the reagents to assay the one or more biomarkers in one or more samples.

### Definitions

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

Throughout this application, various embodiments may be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

As used in the specification and claims, the singular forms “a”, “an” and “the” include plural references unless the context clearly dictates otherwise. For example, the term “a sample” includes a plurality of samples, including mixtures thereof.

The terms “determining,” “measuring,” “evaluating,” “assessing,” “assaying,” and “analyzing” are often used interchangeably herein to refer to forms of measurement. The terms include determining if an element is present or not (for example, detection). These terms can include quantitative, qualitative or quantitative and qualitative determinations. Assessing can be relative or absolute. “Detecting the presence of” can include determining the amount of something present in addition to determining whether it is present or absent depending on the context.

The terms “subject,” “individual,” or “patient” are often used interchangeably herein. A “subject” can be a biological entity containing expressed genetic materials. The biological entity can be an animal. The subject can be tissues, cells and their progeny of a biological entity obtained in vivo or cultured in vitro. The subject can be a mammal. The mammal can be a human. The subject may be diagnosed or suspected of being at high risk for a disease. In some cases, the subject is not necessarily diagnosed or suspected of being at high risk for the disease. A patient described herein, may in some embodiments, be a non-human subject. In some embodiments, the patient is a human. In some embodiments, the patient is female. In some embodiments, the patient is male.

As used herein, the term “about” a number refers to that number plus or minus 10% of that number. The term “about” a range refers to that range minus 10% of its lowest value and plus 10% of its greatest value.

As used herein, the terms “treatment” or “treating” are used in reference to a pharmaceutical or other intervention regimen for obtaining beneficial or desired results in the recipient. Beneficial or desired results include but are not limited to a therapeutic benefit and/or a prophylactic benefit. A therapeutic benefit may refer to eradication or amelioration of symptoms or of an underlying disorder being treated. Also, a therapeutic benefit can be achieved with the eradication or amelioration of one or more of the physiological symptoms associated with the underlying disorder such that an improvement is observed in the subject, notwithstanding that the subject may still be afflicted with the underlying disorder. A prophylactic effect includes delaying, preventing, or eliminating the appearance of a disease or condition, delaying or eliminating the onset of symptoms of a disease or condition, slowing, halting, or reversing the progression of a disease or condition, or any combination thereof. For prophylactic benefit, a subject at risk of developing a particular disease, or to a subject reporting one or more of the physiological symptoms of a disease may undergo treatment, even though a diagnosis of this disease may not have been made.

The section headings used herein are for organizational purposes only and are not to be construed as limiting the subject matter described.

### EXAMPLES

The following examples are included for illustrative purposes only and are not intended to limit the scope of the invention.

### Example 1: Identifying Oxylipin Measurements Indicative of HSD17B13 Activity

An assay is performed to identify oxylipin measurements from Table 1 in blood samples and blood lipid samples comprising lipoproteins. The samples are from human subjects homozygous for inactivating HSD17B13 genotypes such as a TA/TA allele genotype of rs72613567, as well as control subjects homozygous for normal HSD17B13 activity such as a T/T allele genotype of rs72613567. Samples will be obtained from 15 patients in each group (control and homozygous). The measurements are obtained for endogenous oxylipins, as well as isotopic oxylipins measured after addition of isotopically labeled linoleic acid and alpha-linoleic acid. The measurements are assessed to identify oxylipins with significant differences between the control subjects and the subjects with homozygous inactivating HSD17B13 genotypes. These identified oxylipins are used in a panel for assessing HSD17B13 activity. The panel may also be used for identifying liver disease risk based on HSD17B13's association with liver diseases.

### Example 2: Identifying HSD17B13 Inhibitor Target Engagement Using Oxylipin Measurements

The panel from Example 1 is used in an assay to determine HSD17B13 inhibitor target engagement, and to identify an effective dose for target engagement by the HSD17B13 inhibitor. Normal subjects are administered an HSD17B13 inhibitor, and oxylipin measurements from the panel are obtained in samples from the subjects. Each subject is administered a range of doses of the HSD17B13 inhibitor to obtain dose-response curves for the panel of oxylipin measurements. The dose-response curves are compared to the oxylipin measurements of the subjects in Example 1 that are homozygous for inactivating HSD17B13 genotypes to identify an effective dose of the HSD17B13 inhibitor that leads to similar measurements for oxylipins of the panel. Human patients are then recommended to be given that dose going forward.

A dose response curve may also be obtained in individual patients that have a liver disease or that may be at risk of having the liver disease. The dose in each patient that leads to oxylipin measurements that are similar to the oxylipin measurement readouts in the subjects homozygous for the inactivating HSD17B13 genotypes is then the dose that each patient is given or that a dose is adjusted to for each patient.

If complete HSD17B13 inactivation by an HSD17B13 inhibitor is not desired, the dose may be adjusted to levels based on oxylipin measurements obtained in the heterozygous patients. This would be useful for identifying a middle dose that reduces HSD17B13 activity without completely inactivating it, and recommending or providing the middle dose to patients where reduced HSD17B13 activity is desired.

### Example 3: Targeting HSD17B13, a Genetically Validated Target for Chronic Liver Disease, with a Small Molecule Inhibitor in Models of NASH

Polymorphisms rendering the lipid droplet-located protein HSD17B13 enzymatically inactive may protect against NASH, cirrhosis, and liver cancer and have been associated with a reduction in hepatic inflammation and fibrosis. HSD17B13 may oxidize hydroxylated lipid substrates including 12-hydroxyeicosatrienoic acid (12-HETE).

A small molecule HSD17B13 inhibitor was evaluated for inhibition of purified human HSD17B13-oxidation of multiple substrates by quantitation of product formation. Selectivity was determined by biochemical assays for HSD17B family members and broad off target screens. Triplicate co-cultures containing primary human hepatocytes, homozygous for the active HSD17B13 allele, Kupffer cells, and stellate cells were prepared in LC12 microphysiological system (CNBio) in a high fat media with or without the HSD17B13 inhibitor. Fibrosis in the co-culture was analyzed by immunohistochemistry. The HSD17B13 inhibitor was dosed orally in 8-10-week-old Zucker obese rats. HSD17B13 inhibitor concentrations, metabolomics and lipidomics were performed by LC/MS on media from the liver-on-a-chip and plasma from Zucker obese rats.

The HSD17B13 inhibitor inhibited HSD17B13 with low nM potency. The HSD17B13 inhibitor had >100-fold selectivity over other HSD17B family members as well as safety and ADME targets. The HSD17B13 inhibitor treatment of the liver-on-a-chip NASH model led to a decrease in fibrotic proteins with a maximal decrease in mean αSMA (−45.4±17.1%, p<0.0001) and collagen type 1 (−42.3±12.1%, p<0.0001) compared to high fat-treated control. Media metabolomic changes included a decrease in ratio of products to substrates for PLA2, eg, (lysoPC a C18:1+AA)/PC aa C38:5 (39.0±8.4%, p<0.05) consistent with decreased PLA2 activity. Lipidomic changes observed in plasma from Zucker obese rats following repeat dosing of the HSD17B13 inhibitor included a 79-fold increase in the HSD17B13 substrate, 12-HETE, consistent with inhibition of HSD17B13.

The HSD17B13 inhibitor decreased fibrotic marker induction in response to high fat media in primary human liver on a chip consistent with the observation of decreased fibrosis in NASH subjects carrying inactive HSD17B13 alleles. In vivo testing of the HSD17B13 inhibitor testing in Zucker obese rats was supported by metabolic changes in response to repeat oral dosing.

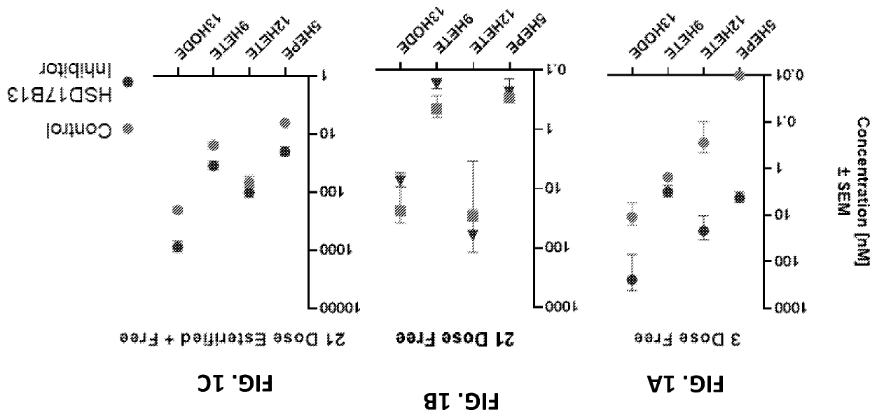

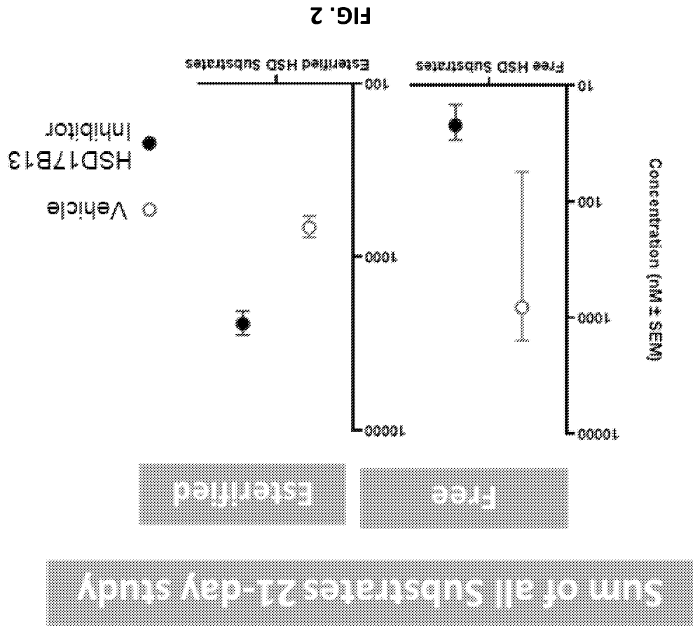

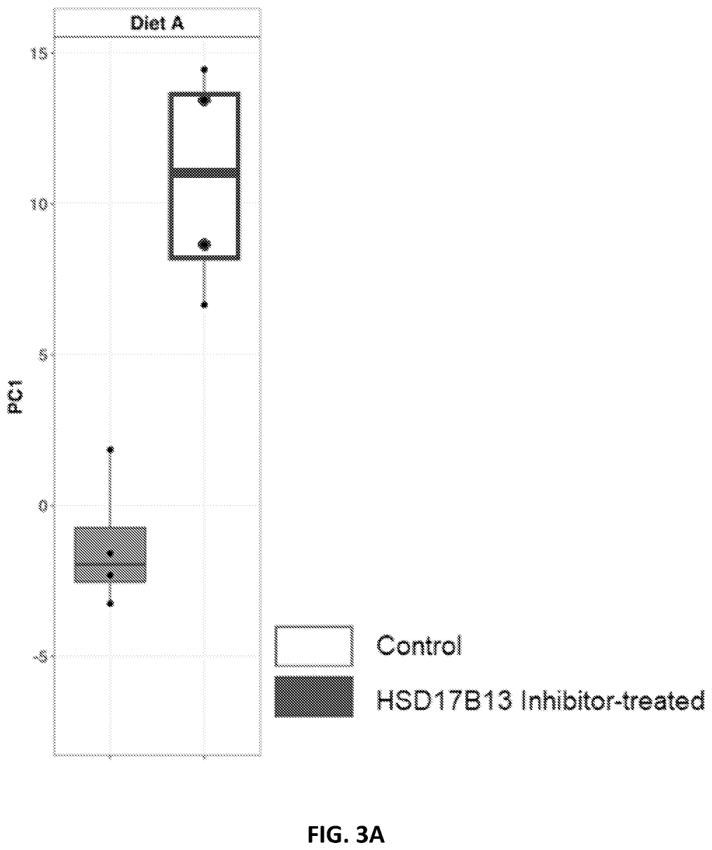

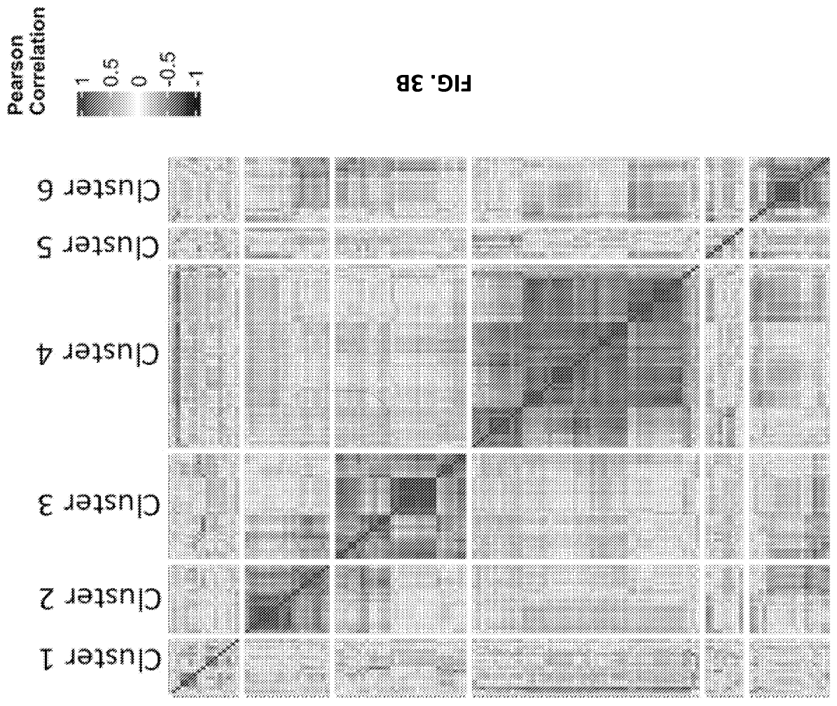

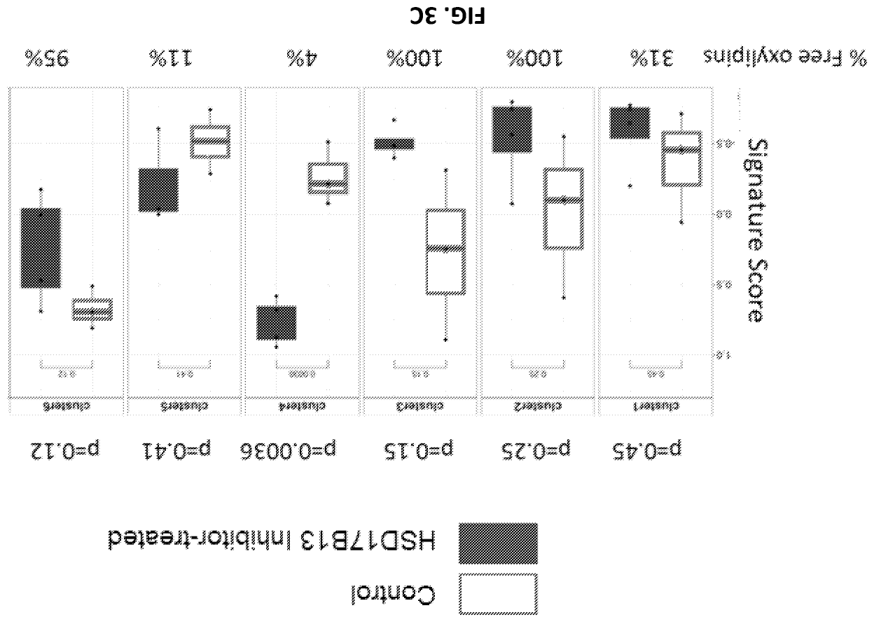

As shown in FIG. 1A-1C and FIG. 2, a HSD17B13 inhibitor increased free substrates acutely and increase esterified substrates following 3 weeks dosing in Zucker obese rats, and as shown in FIG. 3A-3C, the HSD17B13 inhibitor altered bioactive lipids following 3 weeks dosing in the Zucker obese rats on a high fat high cholesterol diet. Oral dosing in Zucker rats increased bioactive oxylipin substrates in the esterified pool. Several known free substrates were acutely elevated, but upon 3-week dosing, esterified 12-HETE and esterified substrates were elevated, and free substrates were decreased compared to plasma from vehicle treated rats. A principal component analysis identified one PC changed in response to the HSD17B13 inhibitor. A hierarchical cluster analysis identified 6 clusters of bioactive lipids that segregated together. Cluster 4 which contained known HSD17B13 substrates was significantly elevated in response to the HSD17B13 inhibitor dosing and was composed dominantly of esterified oxylipins. Dosing with the HSD17B13 inhibitor in Zucker obese rats acutely increased free substrates and increased esterified substrates following 3 weeks of dosing.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A treatment method, comprising:
obtaining or receiving an oxylipin measurement in a biological sample of a subject, wherein the oxylipin comprises an oxylipin other than 13-HODE, 15-HETE, 12-HETE, RvE1, RvD1, LTB3, PDX, and Mar-1; and
based on the oxylipin measurement, administering or discontinuing administration of a 17p-hydroxysteroid dehydrogenase type 13 (HSD17B13) inhibitor to the subject.

2. The method of claim 1, further comprising identifying the subject as a candidate for HSD17B13 inhibitor treatment based on the oxylipin measurement.

3. The method of claim 1, wherein the subject has already received an earlier HSD17B13 inhibitor administration.

4. The method of claim 1, further comprising identifying an effective dose of the HSD17B13 inhibitor based on the oxylipin measurement.

5. The method of claim 1, further comprising identifying a dose of the HSD17B13 inhibitor based on the oxylipin measurement that reproduces a HSD17B13 phenotype in the subject.

6. The method of claim 1, wherein administering the HSD17B13 inhibitor to the subject comprises adjusting a dose of the HSD17B13 inhibitor.

7. The method of claim 1, further comprising evaluating a HSD17B13 inhibitor treatment of the subject based on the oxylipin measurement.

8. The method of claim 1, further comprising evaluating a disease or likelihood of disease development in the subject state based on the oxylipin measurement.

9. The method of claim 1, wherein the oxylipin measurement is elevated or is decreased in the subject, relative to a control or baseline oxylipin measurement.

10. The method of claim 1, wherein the oxylipin comprises 9(10)-EpO, 10-Nitrooleate, 9-Nitrooleate, 9,10-e-DiHO, 9(10)-EpOME, 12(13)-EpOME, 10-Nitrolinoleate, 9-KODE, 13-KODE, 12(13)Ep-9-KODE, 9-HODE, 13-HpODE, 9-HpODE, 9,12,13-TriHOME, 9,10-DiHOME, 12,13-DiHOME, 9(10)-EpODE, 15(16)-EpODE, 12(13)-EpODE, 9-HOTE, 13-HOTE, 9,10-DiHODE, 15,16-DiHODE, 12,13-DiHODE, PGE1, 8,15-DiHETE, 5,15-DiHETE, LTB4, 8(9)-EpETrE, 14(15)-EpETrE, 11(12)-EpETrE, PGD2, 6-keto-PGF1a, 15-keto-PGE2, 15-deoxy PGJ2, PGE2, PGF2a, 15-KETE, 5-KETE, 8-HETE, 9-HETE, 11-HETE, 5-HETE, 20-HETE, LxA4, LxB4, TXB2, 14,15-DiHETrE, 11,12-DiHETrE, 8,9-DiHETrE, 5,6-DiHETrE, RvE2, LTB5, 17(18)-EpETE, 14(15)-EpETE, 11(12)-EpETE, PGE3, PGF3a, 9-HEPE, 5-HEPE, 18-HEPE, 15-HEPE, 12-HEPE, 17,18-DiHETE, 14,15-DiHETE, 19(20)-EpDoPE, 16(17)-EpDoPE, 4-HDoHE, 17-HDoHE, 14-HDoHE, RvD2, 19,20-DiHDoPE, PEA, SEA, OEA, LEA, aLEA, AEA, DEA, PGE2 EA, DHEA, PGF2a EA, PGD2 EA, 15-HETE EA, 11(12)-EpETre EA, 20-COOH-LTB4, 20-OH-LTB4, PGD3, 9,10,13-TriHOME, PGD1, 11,12-,15-TriHETrE, PGJ2, PGB2, 6-trans-LTB4, 11,12-DiHETE, 8,9-DiHETE, 19,20-DiHDPE, 16,17-DiHDPE, 13,14-iHDPE, 9-HOTrE, 10,11-DiHDPE, EKODE, 13-HOTrE, 5,6-DiHETE, 15-deoxy-PGJ2, 7,8-DiHDPE, 8-HEPE, 4,5-DiHDPE, 13-oxo-ODE, 15-oxo-ETE, 9-oxo-ODE, 8(9)-EpETE, 15(S)-HETrE, 12-oxo-ETE, 19(20)-EpDPE, 16(17)-EpDPE, 13(14)-EpDPE, 5-oxo-ETE, 10(11)-EpDPE, 7(8)-EpDPE, 8(9)-EpETrE, alt 5(6)-EpETrE, LTC4, LTD4, or LTE4, or a combination thereof.

11. The method of claim 10, wherein the oxylipin measurement comprises 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, or 132 oxylipin measurements.

12. The method of claim 1, wherein the oxylipin comprises an epoxide oxylipin.

13. The method of claim 1, wherein the oxylipin comprises a hydroxyl oxylipin.

14. The method of claim 1, wherein the oxylipin comprises a keto oxylipin.

15. The method of claim 1, wherein the oxylipin is arachidonic acid-derived.

16. The method of claim 1, wherein the oxylipin is eicosapentaenoic acid-derived.

17. The method of claim 1, wherein the oxylipin is docosahexaenoic acid-derived.

18. The method of claim 1, wherein the oxylipin is linoleic acid-derived.

19. The method of claim 1, wherein the oxylipin is alpha-linoleic acid-derived.

20. The method of claim 1, wherein the oxylipin is endogenous to the subject.

21. The method of claim 1, further comprising administering linoleic acid to the subject prior to obtaining the oxylipin measurement, or wherein the subject has been administered linoleic acid prior to receiving the oxylipin measurement.

22. The method of claim 21, wherein the administered linoleic acid comprises a label.

23. The method of claim 1, further comprising administering alpha-linoleic acid to the subject prior to obtaining the oxylipin measurement, or wherein the subject has been administered alpha-linoleic acid prior to receiving the oxylipin measurement.

24. The method of claim 23, wherein the administered alpha-linoleic acid comprises a label.

25. The method of claim 22 or 24, wherein the label comprises an isotopic label.

26. The method of claim 25, wherein the isotopic label comprises ¹³C or ²H.

27. The method of claim 22 or 24, wherein the oxylipin also comprises the label.

28. The method of claim 1, wherein the oxylipin measurement comprises measurements of at least: 2, 3, 4, 5, 6, 7, 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, or 90 different oxylipins.

29. The method of claim 1, wherein the disease comprises a liver disease.

30. The method of claim 1, wherein the sample comprises a tissue.

31. The method of claim 30, wherein the tissue comprises liver sample.

32. The method of claim 1, wherein the sample comprises a biofluid sample.

33. The method of claim 32, wherein the biofluid comprises blood, serum, or plasma.

34. The method of claim 1, wherein the sample comprises a sample of lipoproteins.

35. The method of claim 34, wherein the lipoproteins comprise low-density lipoproteins (LDLs), very low-density lipoproteins (VLDLs), or high-density lipoproteins (HDLs).

36. The method of claim 34 or 35, wherein the lipoproteins are enriched in the sample.

37. The method of claim 1, wherein the subject is a mammal.

38. The method of claim 37, wherein the subject is a human.
